(19) United States
(12) Patent Application Publication
(10) Pub. No.: US 2022/0362164 A1
(43) Pub. Date: Nov. 17, 2022

(54) METHOD OF TREATMENT AND DEVICE FOR THE IMPROVED BIOAVAILABILITY OF LEUKOTRIENE RECEPTOR ANTAGONISTS

(71) Applicant: INTELGENX CORP., Saint-Laurent (CA)

(72) Inventors: Nadine PAIEMENT, St-Laurent (CA); Horst G. ZERBE, Hudson (CA); Justin W. CONWAY, Carignan (CA); Rodolphe OBEID, St-Laurent (CA)

(73) Assignee: INTELGENX CORP., SAINT-LAURENT (CA)

(21) Appl. No.: 17/729,442

(22) Filed: Apr. 26, 2022

Related U.S. Application Data
(63) Continuation of application No. 15/940,288, filed on Mar. 29, 2018, now abandoned.
Application No. 15/940,288 is a continuation-in-part of application No. 15/067,309, filed on Mar. 11, 2016, now abandoned.
Application No. 15/067,309 is a continuation-in-part of application No. 15/299,054, filed on Oct. 20, 2016, now Pat. No. 9,949,934.
(60) Provisional application No. 62/478,876, filed on Mar. 30, 2017.

(51) Int. Cl. A61K 9/70; A61K 31/47; A61K 47/10; A61K 47/14; A61K 47/18; A61K 9/00

(52) U.S. Cl. CPC A61K 9/7007; A61K 31/47; A61K 47/10; A61K 47/14; A61K 47/183; A61K 9/006

(57) ABSTRACT

Disclosed is a method of administration and device for the improved bioavailability of leukotriene receptor antagonists. This method and device involve an alkaline surface pH oral film dosage form designed to deliver leukotriene receptor antagonists, such as Montelukast, to the stomach in an amorphous precipitate suspended in aqueous medium. Also disclosed is a device and method for treating a disease, such as a neurodegenerative disease or condition associated with neuroinflammation induced by a leukotriene. The device is a film unit dosage form having an alkaline surface pH film layer and a safe and effective amount of Montelukast. The device is configured and formulated to predominantly achieve enteral delivery of the Montelukast. The method includes enterally delivering to a human or an animal in need of treatment, a safe and effective amount of Montelukast capable of crossing the blood-brain barrier.

### CROSS-REFERENCE TO RELATED APPLICATIONS

The present application is a continuation of U.S. application Ser. No. 15/940,288, filed on Mar. 29, 2018, and which claims priority to Provisional Application No. 62/478,876, filed Mar. 30, 2017, and which also is a continuation-in-part of U.S. application Ser. No. 15/067,309, filed Mar. 11, 2016; and Ser. No. 15/299,054, filed Oct. 20, 2016. These documents are hereby incorporated by reference in their entireties.

### FIELD OF THE DISCLOSURE

This disclosure concerns a formulation and method of treatment and pharmaceutical dosage form for improving the bioavailability of a leukotriene receptor antagonist or leukotriene synthesis inhibitor for the treatment of a disorder.

### BACKGROUND OF THE DISCLOSURE

As the brain ages, it loses its ability to generate new cells, while existing cells lose functionality, including the ability to prevent inflammatory mediators in the blood from passing through the blood-brain barrier (BBB). At the same time the aged brain tends to produce higher levels of inflammatory agents such as leukotrienes, and loses some of its ability to counter the effects of inflammatory mediators, resulting in neuroinflammation and cognitive impairment. A major contributor to neuroinflammation are leukotrienes. There is evidence that leukotriene receptor antagonists, such as Montelukast sodium, have the potential to reduce neuroinflammation and restore brain cell function. Such treatments can be effective for treating various neurodegenerative diseases and conditions, including Huntington's disease, Parkinson's disease, loss of memory function, spinal cord and brain injuries, and stroke.

Montelukast (MTL) sodium is an orally active leukotriene receptor antagonist commonly used to treat patients suffering from chronic asthma as well as symptomatic relief of seasonal allergic rhinitis. During a normal respiratory inflammation response, the binding of cysteinyl leukotrienes to the leukotriene receptor induces inflammation within the respiratory pathway, generating asthmatic symptoms. MTL functions to suppress this inflammatory response by binding to the leukotriene receptor with high affinity and selectivity, thereby blocking the pathway leading to the physiological response for extended periods. Recently, neuroinflammation within the brain has been linked to age-related dementia and neurodegenerative diseases. MTL applied under these biological conditions has been shown to significantly reduce neuroinflammation, elevate hippocampal neurogenesis and improve learning and memory in old animals.

Presently, Montelukast sodium is marketed in a tablet form under the name of “Singulair®.” One of the greatest challenges for using MTL in a tablet form is the inconsistent bioavailability. Although MTL is freely soluble in water, its solubility is reduced under acidic conditions normally found in the stomach. This has led to relatively slow and inconsistent absorption into the blood stream, with maximum concentrations occurring only after 2-4 hours, thereby limiting its use to chronic applications rather than for rapid acute treatment. Experimental studies indicate that the major obstacles limiting MTL absorption pertain to its solubility, the rate of dissolution from the tablet platform and the rate of transport/permeation across biological membranes.

U.S. Pat. Nos. 8,575,194 and 9,149,472 disclose methods of improving cognitive impairments by administering Montelukast in a single tablet or capsule that comprises an extended release (ER) component and an immediate release (IR) component in a single dosage unit. The method involves administering the dosage unit to provide an initial burst of IR active pharmaceutical ingredient (API) into the system, followed by the ER API over the course of 12 hours, thereby maintaining a constant effective plasma level. Disclosed embodiments include a tablet with an ER core and an IR shell or a capsule containing a mixture of ER and IR beads combined in a specific ratio to achieve the desired effect. In an alternative embodiment, the regimen in general consists of an initial high dose of 10 mg of MTL followed by 5 mg doses approximately every 2 hours afterwards over the course of 12 hours. The patents discuss plasma levels as being critical for achieving cognitive improvement.

However, MTL can only exert its therapeutic effects if it crosses the blood-brain barrier (BBB) and accumulates in the cerebrospinal fluid (CSF) at sufficient concentration levels. Neither plasma nor CSF concentration levels of MTL are discussed in the patents.

Moreover, pharmacokinetics research (http://www.accessdata.fda.gov/drugsatfda_do_cs/nda/2000/20830S008_Singulair_biopharmr.pdf) related to MTL CSF concentrations indicates (see page 7 pharmacokinetics research document) that MTL is not expected to cross the BBB as it is more than 99% bound to plasma proteins. In this study rats dosed with radiolabeled MTL exhibited only minimal distribution across the blood-brain barrier.

Surge Dose® Montelukast tablets have been proposed in a method for improving the formulation of a tablet capable of accelerated API release. The method attempts to improve MTL solubility in the stomach. The Surge Dose® product may still be limited by gastric emptying cycles and food effects similar to the Singulair® tablet and chewable. The chewable tablet is also comprised of solid MTL.

There is thus a need for method of treatment that overcome the shortcomings of the prior art.

### SUMMARY OF THE DISCLOSURE

Disclosed is an alkaline oral film dosage form for improving bioavailability of leukotriene antagonist inhibitor. Accordingly, the oral film dosage form deliver leukotriene antagonist inhibitor such as Montelukast in a form that renders it suitable for improved bioavailability when compared with commercially available oral dosage forms. The disclosed oral film dosage form has an alkaline surface pH that is preferably greater than 7.5, more preferably greater than 8 and optimally greater than 8.5.

Disclosed is a dosage form of a leukotriene receptor antagonist exhibiting an improved bioavailability as compared with existing oral dosage forms.

Disclosed is an exemplary dosage form exhibiting an improved bioavailability of Montelukast leukotriene receptor antagonist.

Disclosed is a dosage form for delivering to the brain a safe and effective amount of leukotriene receptor antagonists for reducing neuroinflammation.

Disclosed is an exemplary dosage form for delivering to the brain a safe and effective amount of Montelukast for reducing neuroinflammation.

Disclosed is a pharmaceutical dosage form for human pharmaceutical use, comprising Montelukast salt, free base, or prodrug in a unit dosage form suitable for oral administration. The dosage form can be configured for enteral delivery of the active agent. The Montelukast salt, free base, or prodrug according to the disclosed dosage form can be configured to reach the stomach in an amorphous form in aqueous suspension.

Disclosed is Montelukast solubilized in an oral dosage form. The oral dosage form can be orally administered, such as on the tongue, buccaly or sublingually. Upon contact of the dosage form with saliva, the dosage form preferably solubilizes and/or disintegrates. The dissolution and/or disintegration of the oral dosage form transforms the solubilized Montelukast into a suspended and/or insoluble precipitate creating a pre-solubilized dosage form ready to be absorbed and/or swallowed in the oral cavity.

According to an aspect of the present disclosure, the pre-solubilized dosage form improves the bioavailability of the Montelukast compared with the equivalent tablet or chewable oral dosage forms.

The Montelukast may be delivered through the use of a film layer having an alkaline surface pH. As such, Montelukast salt, free base, or prodrug is disposed within or on a polymeric film suitable for oral administration. The films can be formulated for rapid disintegration and distribution of micro- or nano-scopic particles of the active agent in the gastrointestinal tract.

In certain embodiments, the active agent in the film dosage form is Montelukast sodium.

According to an aspect of the present disclosure, there is provided a alkaline surface pH Montelukast oral film dosage form having an improved bioavailability when compared to swallowable and chewable oral tablet dosage forms.

Also disclosed is a method of treating conditions where leukotriene inhibition is desired (e.g., either by blocking leukotriene receptors or by inhibiting leukotriene synthesis), which comprises administering to a patient in need thereof an oral film dosage form having an alkaline surface pH and containing about 0.5 to about 25 mg of Montelukast, as needed, up to a total dose of 25 mg per day for the treatment of neuroinflammation.

Specific conditions that can be treated by the present disclosure, include, but are not limited to, neuroinflammation, neurodegenerative diseases and cognitive impairment.

In particular, the present disclosure is directed to a pharmaceutical unit dosage composition comprising about 0.5 to about 25 mg of Montelukast.

The unit dosage form is suitable for oral administration to treat neuroinflammation. The unit dosage form contains about 10 mg of the compound and is administered once or twice per day.

Also disclosed is a method of treating a neurodegenerative disease or neuroinflammatory disorder. The method comprising the steps of enterally delivering to a person or other animal in need of treatment for a neurodegenerative disease or neuroinflammatory disorder via a film dosage form, a safe and effective amount of a leukotriene receptor antagonist, wherein the amount of Montelukast is about 0.5 mg to about 25 mg per day, preferably about 1 mg to about 10 mg and wherein leukotriene receptor antagonist is enterally delivered as a precipitate suspended in an aqueous medium, wherein the precipitate is generated orally upon dissolution and/or disintegration of an oral film dosage form

Also disclosed is an oral film dosage form, comprising: a film layer having an alkaline surface pH; and a safe and effective amount of a leukotriene receptor antagonist incorporated into the film layer. The film layer is formulated to dissolve and/or disintegrate when in contact with an aqueous solution. The leukotriene receptor antagonist is preferably incorporated into the film layer in an amorphous form and most preferably solubilized in the film layer. A preferred film dosage form comprises Montelukast, present in an amount of about 0.5 mg to about 25 mg, preferably about 5 mg to about 15 mg and most preferably about 10 mg.

Also disclosed is an oral film dosage form having a film layer with an alkaline surface pH; and a safe and effective amount of a leukotriene receptor antagonist incorporated into the film layer wherein the film layer dissolves and/or disintegrates in contact with an aqueous solution. The alkalinity of the surface is preferably greater than pH 7.5, more preferably greater than pH 8.0 and most preferably greater than pH 8.5. Also disclosed is an oral dosage form having an unbuffered alkaline surface pH.

Also disclosed is an oral film dosage form having a film layer with an alkaline surface pH; and a safe and effective amount of a leukotriene receptor antagonist incorporated into the film layer wherein the film layer dissolves and/or disintegrates in contact with an aqueous solution, and wherein the film layer comprises a plurality of stabilizers. The plurality of stabilizers may be selected from parabens, EDTA, BHT and combinations of parabens, EDTA and BHT.

Also disclosed is a film dosage form comprising Montelukast, wherein the area under the curve (AUC) is between about 3120 and about 4700 ng*h/mL and/or wherein the Cmax is between about 475 and about 720 ng/mL.

Also disclosed is a method of treating neurodegenerative diseases and conditions at least partially induced by leukotrienes, by administering to a person or other animal in need of treatment, a film dosage form including a film layer comprising Montelukast. The film layer(s) is configured for enteral delivery of the active agent.

The film layer may also be configured for transmucosal or sublingual delivery.

These and other features, advantages and objects of the various embodiments will be better understood with reference to the following specification and claims.

### BRIEF DESCRIPTION OF THE DRAWINGS

FIG. 1 is representation of the dissolution of swallowable tablets.

FIG. 2 is an illustrative representation of the absorption or an oral film dosage form when administered to a subject.

FIG. 3 is an illustrative representation of the behavior of the active in the stomach following administration of the oral film to a subject.

FIG. 4 is illustrative representation of the transmucosal absorption following administration of the oral film to a subject.

FIG. 5 is a graphical representation of the dissolution data shown in Table 12.

FIG. 6 is a graphical representation of the solubility limits of MTL in solutions containing EDTA.

### DETAILED DESCRIPTION OF ILLUSTRATIVE EMBODIMENTS

In accordance with certain aspects of this disclosure, methods of administration and devices for the improved bioavailability of leukotriene inhibitors are provided. This method and device involve an oral dosage form designed to deliver leukotriene inhibitors such as Montelukast, to the mouth and stomach in the form of an amorphous precipitate suspended in an aqueous medium (e.g., saliva and/or gastric fluids).

In accordance with certain aspects of this disclosure, methods for treating neurodegenerative diseases and/or other conditions that are at least partially induced by leukotrienes are provided. These methods include enteral delivery or a combination of transmucosal, sublingual or both transmucosal and sublingual, along with enteral delivery of Montelukast. The Montelukast is incorporated into a film layer in an amount that is safe and effective to reduce leukotriene induced neuroinflammation in patients.

Neurodegenerative diseases that can be treated in accordance with this disclosure include, but are not limited to, loss of memory function (long term or short term), dementia, apathy, depression, fatigue (acute or chronic), cognitive losses, loss of focus, loss of libido, and disorientation. Specific disease conditions that can be treated with the disclosed methods include Huntington's disease, Parkinson's disease and Alzheimer's disease. Such treatments can also be effective for treating neurological diseases, neurodegenerative diseases, neuroinflammatory disorders, traumatic or posttraumatic disorders, vascular or more precisely, neurovascular disorders, hypoxic disorders, and postinfectious central nervous system disorders. The term “neurodegenerative disease” or “neurological disease” or “neuroinflammatory disorder” refers to any disease, disorder, or condition affecting the central or peripheral nervous system, including ADHD, AIDS-neurological complications, absence of the Septum Pellucidum, acquired epileptiform aphasia, acute disseminated encephalomyelitis, adrenoleukodystrophy, agenesis of the Corpus Callosum, agnosia, Aicardi Syndrome, Alexander Disease, Alpers' Disease, alternating hemiplegia, Alzheimer's Disease, amyotrophic lateral sclerosis (ALS), anencephaly, aneurysm, Angelman Syndrome, angiomatosis, anoxia, aphasia, apraxia, arachnoid cysts, arachnoiditis, Arnold-Chiari Malformation, arteriovenous malformation, aspartame, Asperger Syndrome, ataxia telangiectasia, ataxia, attention deficit-hyperactivity disorder, autism, autonomic dysfunction, back pain, Barth Syndrome, Batten Disease, Behcet's Disease, Bell's Palsy, benign essential blepharospasm, benign focal amyotrophy, benign intracranial hypertension, Bernhardt-Roth Syndrome, Binswanger's Disease, blepharospasm, Bloch-Sulzberger Syndrome, brachial plexus birth injuries, brachial plexus injuries, Bradbury-Eggleston Syndrome, brain aneurysm, brain injury, brain and spinal tumors, Brown-Sequard Syndrome, bulbospinal muscular atrophy, Canavan Disease, Carpal Tunnel Syndrome, causalgia, cavernomas, cavernous angioma, cavernous malformation, central cervical cord syndrome, central cord syndrome, central pain syndrome, cephalic disorders, cerebellar degeneration, cerebellar hypoplasia, cerebral aneurysm, cerebral arteriosclerosis, cerebral atrophy, cerebral beriberi, cerebral gigantism, cerebral hypoxia, cerebral palsy, cerebro-oculo-facio-skeletal syndrome, Charcot-Marie-Tooth Disorder, Chiari Malformation, chorea, choreoacanthocytosis, chronic inflammatory demyelinating polyneuropathy (CIDP), chronic orthostatic intolerance, chronic pain, Cockayne Syndrome Type II, Coffin Lowry Syndrome, coma, including persistent vegetative state, complex regional pain syndrome, congenital facial diplegia, congenital myasthenia, congenital myopathy, congenital vascular cavernous malformations, corticobasal degeneration, cranial arteritis, craniosynostosis, Creutzfeldt-Jakob Disease, cumulative trauma disorders, Cushing's Syndrome, cytomegalic inclusion body disease (CIBD), cytomegalovirus infection, dancing eyes-dancing feet syndrome, Dandy-Walker Syndrome, Dawson Disease, De Morsier's Syndrome, Dejerine-Klumpke Palsy, delir in elderly, trauma-induced delir, dementia-multi-infarct, dementia-subcortical, dementia with Lewy Bodies, dermatomyositis, developmental dyspraxia, Devic's Syndrome, diabetic neuropathy, diffuse sclerosis, Dravet's Syndrome, dysautonomia, dysgraphia, dyslexia, dysphagia, dyspraxia, dystonias, early infantile epileptic encephalopathy, Empty Sella Syndrome, encephalitis lethargica, encephalitis and meningitis, encephaloceles, encephalopathy, encephalotrigeminal angiomatosis, epilepsy, Erb's Palsy, Erb-Duchenne and Dejerine-Klumpke Palsies, Fabry's Disease, Fakir's Syndrome, fainting, familial dysautonomia, familial hemangioma, familial idiopathic basal ganglia calcification, familial spastic paralysis, febrile seizures (e.g., GEFS and GEFS plus), Fisher Syndrome, Floppy Infant Syndrome, Friedreich's Ataxia, Gaucher's Disease, Gerstmann's Syndrome, Gerstmann-Straussler-Scheinker Disease, giant cell arteritis, giant cell inclusion disease, globoid cell leukodystrophy, glossopharyngeal neuralgia, Guillain-Barre Syndrome, HTLV-1 associated myelopathy, Hallervorden-Spatz Disease, head injury, headache, hemicrania continua, hemifacial spasm, hemiplegia alterans, hereditary neuropathies, hereditary spastic paraplegia, heredopathia atactica polyneuritiformis, Herpes Zoster Oticus, Herpes Zoster, Hirayama Syndrome, holoprosencephaly, Huntington's Disease, hydranencephaly, hydrocephalus-normal pressure, hydrocephalus, hydromyelia, hypercortisolism, hypersomnia, hypertonia, hypotonia, hypoxia, immune-mediated encephalomyelitis, inclusion body myositis, incontinentia pigmenti, infantile hypotonia, infantile phytanic acid storage disease, infantile refsum disease, infantile spasms, inflammatory myopathy, intestinal lipodystrophy, intracranial cysts, intracranial hypertension, Isaac's Syndrome, Joubert Syndrome, Kearns-Sayre Syndrome, Kennedy's Disease, Kinsboume syndrome, Kleine-Levin syndrome, Klippel Feil Syndrome, Klippel-Trenaunay Syndrome (KTS), Klüver-Bucy Syndrome, Korsakoff s Amnesic Syndrome, Krabbe Disease, Kugelberg-Welander Disease, kuru, Lambert-Eaton Myasthenic Syndrome, Landau-Kleffner Syndrome, lateral femoral cutaneous nerve entrapment, lateral medullary syndrome, learning disabilities, Leigh's Disease, Lennox-Gastaut Syndrome, Lesch-Nyhan Syndrome, leukodystrophy, Levine-Critchley Syndrome, Lewy Body Dementia, lissencephaly, locked-in syndrome, Lou Gehrig's Disease, lupus-neurological sequelae, Lyme Disease-Neurological Complications, Machado-Joseph Disease, macrencephaly, megalencephaly, Melkersson-Rosenthal Syndrome, meningitis, Menkes Disease, meralgia paresthetica, metachromatic leukodystrophy, microcephaly, migraine, Miller Fisher Syndrome, mini-strokes, mitochondrial myopathies, Mobius Syndrome, monomelic amyotrophy, motor neuron diseases, Moyamoya Disease, mucolipidoses, mucopolysaccharidoses, multi-infarct dementia, multifocal motor neuropathy, multiple sclerosis (MS), multiple systems atrophy (MSA-C and MSA-P), multiple system atrophy with orthostatic hypotension, muscular dystrophy, myasthenia-congenital, myasthenia gravis, myelinoclastic diffuse sclerosis, myoclonic encephalopathy of infants, myoclonus, myopathy-congenital, myopathy-thyrotoxic, myopathy, myotonia congenita, myotonia, narcolepsy, neuroacanthocytosis, neurodegeneration with brain iron accumulation, neurofibromatosis, neuroleptic malignant syndrome, neurological complications of AIDS, neurological manifestations of Pompe Disease, neuromyelitis optica, neuromyotonia, neuronal ceroid lipofuscinosis, neuronal migration disorders, neuropathy-hereditary, neurosarcoidosis, neurotoxicity, nevus cavernosus, Niemann-Pick Disease, O'Sullivan-McLeod Syndrome, occipital neuralgia, occult spinal dysraphism sequence, Ohtahara Syndrome, olivopontocerebellar atrophy, opsoclonus myoclonus, orthostatic hypotension, Overuse Syndrome, pain-chronic, paraneoplastic syndromes, paresthesia, Parkinson's Disease, parmyotonia congenita, paroxysmal choreoathetosis, paroxysmal hemicrania, Parry-Romberg, Pelizaeus-Merzbacher Disease, Pena Shokeir II Syndrome, perineural cysts, periodic paralyses, peripheral neuropathy, periventricular leukomalacia, persistent vegetative state, pervasive developmental disorders, phytanic acid storage disease, Pick's Disease, Piriformis Syndrome, pituitary tumors, polymyositis, Pompe Disease, porencephaly, Post-Polio Syndrome, postherpetic neuralgia, postinfectious encephalomyelitis, postural hypotension, postural orthostatic tachycardia syndrome, postural tachycardia syndrome, primary lateral sclerosis, prion diseases, progressive hemifacial atrophy, progressive locomotor ataxia, progressive multifocal leukoencephalopathy, progressive sclerosing poliodystrophy, progressive supranuclear palsy, pseudotumor cerebri, pyridoxine dependent and pyridoxine responsive siezure disorders, Ramsay Hunt Syndrome Type I, Ramsay Hunt Syndrome Type II, Rasmussen's Encephalitis and other autoimmune epilepsies, reflex sympathetic dystrophy syndrome, refsum disease-infantile, refsum disease, repetitive motion disorders, repetitive stress injuries, restless legs syndrome, retrovirus-associated myelopathy, Rett Syndrome, Reye's Syndrome, Riley-Day Syndrome, SUNCT headache, sacral nerve root cysts, Saint Vitus Dance, Salivary Gland Disease, Sandhoff Disease, Schilder's Disease, schizencephaly, seizure disorders, septo-optic dysplasia, severe myoclonic epilepsy of infancy (SMEI), shaken baby syndrome, shingles, Shy-Drager Syndrome, Sjogren's Syndrome, sleep apnea, sleeping sickness, Soto's Syndrome, spasticity, spina bifida, spinal cord infarction, spinal cord injury, spinal cord tumors, spinal muscular atrophy, spinocerebellar atrophy, Steele-Richardson-Olszewski Syndrome, Stiff-Person Syndrome, striatonigral degeneration, stroke, Sturge-Weber Syndrome, subacute sclerosing panencephalitis, subcortical arteriosclerotic encephalopathy, Swallowing Disorders, Sydenham Chorea, syncope, syphilitic spinal sclerosis, syringohydromyelia, syringomyelia, systemic lupus erythematosus, Tabes Dorsalis, Tardive Dyskinesia, Tarlov Cysts, Tay-Sachs Disease, temporal arteritis, tethered spinal cord syndrome, Thomsen Disease, thoracic outlet syndrome, thyrotoxic myopathy, Tic Douloureux, Todd's Paralysis, Tourette Syndrome, transient ischemic attack, transmissible spongiform encephalopathies, transverse myelitis, traumatic brain injury, tremor, trigeminal neuralgia, tropical spastic paraparesis, tuberous sclerosis, vascular erectile tumor, vasculitis including temporal arteritis, Von Economo's Disease, Von Hippel-Lindau disease (VHL), Von Recklinghausen's Disease, Wallenberg's Syndrome, Werdnig-Hoffinan Disease, Wernicke-Korsakoff Syndrome, West Syndrome, Whipple's Disease, Williams Syndrome, Wilson's Disease, X-Linked Spinal and Bulbar Muscular Atrophy, and Zellweger Syndrome.

The disclosed dosage forms and methods are expected to be especially useful for treating neurodegenerative diseases and neuroinflammatory disorders selected from the group comprising or consisting of: Alzheimer's disease, Parkinson's disease, Creutzfeldt Jakob disease (CJD), new variant of Creutzfeldt Jakobs disease (rivCJD), Hallervorden Spatz disease, Huntington's disease, multisystem atrophy, dementia, frontotemporal dementia, motor neuron disorders of multiple spontaneous or genetic background, amyotrophic lateral sclerosis (ALS), spinal muscular atrophy, spinocerebellar atrophies (SCAs), schizophrenia, affective disorders, major depression, meningoencephalitis, bacterial meningoencephalitis, viral meningoencephalitis, CNS autoimmune disorders, multiple sclerosis (MS), acute ischemic/hypoxic lesions, stroke, CNS and spinal cord trauma, head and spinal trauma, brain traumatic injuries, arteriosclerosis, atherosclerosis, microangiopathic dementia, Binswanger' disease (Leukoaraiosis), cochlear degeneration, cochlear deafness, AIDS-related dementia, fragile X-associated tremor/ataxia syndrome (FXTAS), progressive supranuclear palsy (PSP), striatonigral degeneration (SND), olivopontocerebellear degeneration (OPCD), Shy Drager syndrome (SDS), age dependant memory deficits, neurodevelopmental disorders associated with dementia, Down's Syndrome, synucleinopathies, superoxide dismutase mutations, trinucleotide repeat disorders as Huntington's Disease, trauma, hypoxia, vascular diseases, vascular inflammations, CNS-ageing. Also age dependent decrease of stem cell renewal may be addressed.

The disclosed dosage forms and methods are expected to be especially useful for treating neurodegenerative diseases and neuroinflammatory disorders selected from the group comprising or consisting of: Alzheimer's disease, Parkinson's disease, Huntington's disease, amyotrophic lateral sclerosis (ALS), hydrocephalus, CNS and spinal cord trauma such as spinal cord injury, head and spinal trauma, brain traumatic injuries, cochlear deafness, AIDS-related dementia, trinucleotide repeat disorders as Huntington's Disease, and CNS-aging.

The words “treatment”, “treating” and variations thereof refer to curing, mitigating or relieving symptoms of a disease, medical condition or injury.

As used herein, a film layer that is “unbuffered” is a film layer that does not contain a weak acid or weak base that is effective to maintain pH near a chosen value upon addition of another acid or base. Stated differently, the unbuffered film layer does not contain a buffering agent, such as borates, citrates, or phosphates.

Enteral delivery refers to passing the active agent through the gastrointestinal tract, either naturally via the mouth and esophagus, or through an artificial opening (e.g., stoma) and absorbing the active agent in the intestine.

Leukotriene inhibitions include leukotriene receptor antagonists and/or leukotriene synthesis inhibitors that block 5-lipoxygenase activity. Such leukotriene inhibitors include, but are not necessarily limited to, leukotriene receptor antagonist such as Montelukast, Zafirlukast, Pranlukast, cinalukast, probilukast, iralukast and sulukast. Active agents capable of existing in various forms, such as base form, salts, esters, prodrugs, etc., are, unless otherwise indicated, encompassed by reference to the base drug. For example, the term “Montelukast” is intended to encompass all forms, including salts (e.g., Montelukast sodium), esters and prodrugs.

The term “amorphous” refers to a non-crystalline form of the solid i.e. a state that lacks the regular crystalline organization of atoms. Amorphous solids are generally more soluble, faster dissolving, easier to absorb in the GI tract or oral cavity and less stable than their crystalline counterparts. The amorphous content (amorphicity) of a solid can be accurately and precisely assessed using a number of well-established methodologies, including isothermal calorimetry, Powder X-ray diffraction (PXRD), Raman Spectroscopy, Differential Scanning calorimetry (DSC), Continuous Relative Humidity Perfusion Microcalorimetry (cRHp), and Dynamic Vapor Sorption (DVS). In this document, the term amorphous also refers to an active agent(s) that exhibits 30% or more than 30% of amorphous material, more preferably above 50%.

The term “active agent(s)” or API (active pharmaceutical ingredient) refers mainly to pharmaceutically active ingredients, but may also refer to generally any agent(s) that chemically interacts with the subject to which it is administered to cause a biological change, such as, but not limited to eliminating symptoms of disease or regulating biological functions.

The term “stable” refers to a product which exhibit no or very limited changes in the dissolution profile and recovery (or assay) when the product is exposed to normal stability conditions (example 25° C./60% RH and 40° C./75% RH) for extended period of time.

An “oral film dosage form” generally refers to an edible composition that can be ingested by a subject (human or animal) to orally, buccally or sublingually administer a predetermined amount of an active agent(s) to the subject, wherein the composition is in the form of a film.

The “surface pH” is the pH measured on a surface of the film, such as the top or bottom surface of a monolayer film or on an exposed surface of the layer containing the active in a multilayer oral film. The film is prepared for pH testing by slightly wetting the film (adding water as needed for a pH test e.g. one to three drops). The pH is then measured by bringing the electrode in contact with the surface of the oral film. This measurement of the surface pH is preferably performed on several films of the same formulation.

The terms “film” and “film layer” refer to a component or layer of a dosage form that is distinctly different from pills, tablets, caplets, and capsules, and in which the dosage form is a thin strip of material. Such films are typically rapidly disintegrating or rapidly dissolving, but can also exhibit longer disintegration and/or dissolution time when required. The films are generally sufficiently flexible to allow bending or even folding without breaking. A film layer is a sheet-like material having a thickness that is much less than its length or width. For example, oral transmucosal devices typically have a thickness on the order of about 50 μm to 500 μm (i.e., 0.05 mm to 0.5 mm), although thicker or thin films may be suitable; and width and length dimensions typically on the order of about 5 mm to 40 mm, although larger or smaller dimensions can be used.

Throughout this disclosure, unless otherwise indicated, it will be appreciated that specific reference to “MTL” or “Montelukast” implies that other leukotriene receptor antagonists may be substituted.

The film dosage form can comprise a single film layer, or multiple layers. For example, in the case of buccal or sublingual film dosage forms, it can be beneficial to employ a biocompatible layer (e.g., a bioadhesive layer) containing the active agent and a non-adhesive barrier layer to prevent or reduce ingestion of the active agent(s) and ensure that all or most of the active agent crosses the mucous membrane to which the bioadhesive layer is applied. The term “bioadhesive” means that the composition of the film layer is formulated to adhere to the selected mucous membrane through which delivery of the active agent is targeted, and encompasses the term “mucoadhesive.” For example, bioadhesive polymers used in formulating the film should be selected to exhibit adequate adhesion within the environment at the targeted mucous membrane to ensure that the bioadhesive layer remains in contact with the mucous membrane to which it is applied and allows the active agent to directly enter the blood stream through the mucous membrane.

The active agent can be combined or blended with film forming polymers and/or bioadhesive polymers to obtain a balanced combination of properties like flexibility, tensile strength, uniformity of the film and the drug, hydration speed, drug release, disintegration time, palatability (taste, smell, texture and aftertaste), mouth feel, mucoadhesion, and chemical and physical stability suitable for an oral delivery device.

Examples of suitable film forming polymers exhibiting bioadhesion include hydroxypropyl cellulose, hydroxymethylcellulose, natural or synthetic gum, polyvinyl alcohol, polyethylene oxide, homo- and copolymers of acrylic acid crosslinked with a polyalkenyl polyether or divinyl alcohol, polyvinylpyrrolidone, hydroxypropylmethyl cellulose, sodium alginate, pectin, gelatin maltodextrins chitosan, and poly-lysines. In certain embodiments or aspects of this disclosure, the active agent can be combined with film forming neutral polysaccharides such as pullulan.

Penetration enhancing agents can also or alternatively be employed to further increase the rate and/or total amount of absorption of the active agent. Examples of penetration enhancers that can be advantageously employed include 2,3-lauryl ether, phosphatidylcholine, aprotinin, polyoxyethylene, azone, polysorbate 80, benzalkonium chloride, polyoxyethylene, cetylpyridinium chloride, phosphatidylcholine, cetyltrimethyl ammonium bromide, sodium EDTA, cyclodextrin, chitosan, sodium glycocholate, dextran sulfate 16 sodium glycodeoxycholate. Other penetration enhancers include surfactants, bile salts (by extracting membrane protein or lipids, by membrane fluidization, by producing reverse micellization in the membrane and creating aqueous channels), fatty acids (that act by disrupting intercellular lipid packing), azone (by creating a region of fluidity in intercellular lipids), pore forming agents (e.g., molecules, peptides, nucleic acids or particles that insert into the lipid membrane and create a hole through which the API can pass) and alcohols (by reorganizing the lipid domains and by changing protein conformation), sulphoxides (dimethylsulphoxide, decylmethyl sulfoxide), pyrrolidones (2pyrrolidone, 2P), alcohols/alkanols (ethanol or decanol), glycols (propylene glycol), terpenes (1,8-cineole, menthol, and menthone, D-limonene), fatty acids (oleic acid, sodium caprate), and bile salts (sodium deoxycholate, sodium deoxyglycocholate). It was found that the permeation and absorption is greatly enhanced through the use of a single or combination of penetration enhancers present in the formulation in the range of 0.05-8.00% dry w/w.

Examples of Anti-oxidants and chelating agents that can be advantageously employed comprise disodium-EDTA, sodium calcium EDTA, citric acid, L-cystein, vitamin E, ascorbyl palmitate, butylated hydroxyanisole, butylated hydroxytoluene, potassium metabisulfite, propyl gallate, sodium metabisulfite, sodium thiosulfate, 3,4-dihydroxybenzoic acid.

Examples of surfactants that can be employed to enhance penetration and/or wettability of the film to promote adhesion, include polysorhates (Tween™, Span™), sodium dodecyl sulfate (sodium lauryl sulfate), lauryl dimethyl amine oxide, cetyltrimethylammonium bromide (CTAB), polyethoxylated alcohols, polyoxyethylene sorbitan octoxynol (Triton X100™), N,N-dimethyldodecylamine-N-oxide, hexadecyltrimethylammonium bromide (HTAB), polyoxyl 10 lauryl ether, Brij 721™, bile salts (sodium deoxycholate, sodium cholate) polyoxyl castor oil (Cremophor™), nonylphenol ethoxylate (Tergitol™), cyclodextrins, lecithin, methylbenzethonium chloride (Hyamine™).

The solubility and disintegration profiles of the film can influence the bioavailability of the drug. Therefore, certain embodiments of the film platform will contain specific quantities of disintegrants to control the residence time of the film in the oral cavity. Certain forms of the drug product may contain between 0-10% by mass of a disintegrant. Examples of disintegrants that could be used are Maltodextrin, Citric acid, Sodium starch, glycolate, crosslinked polyvinylpyrrolidone (crospovidone), crosslinked sodium carboxymethyl cellulose, Calcium silicate, Alginic acid, and vinylpyrrolidonc-vinyl acetate copolymers.

The term “pre-solubilized” as used herein refers to a dosage form comprising an active agent that undergoes a phase transformation in the oral cavity upon administration. For instance, a pre-solubilized form of MTL could be a precipitated MTL previously administered as a solubilized MTL in a film matrix. The pre-solubilized precipitate is not dissolved, but is in a form (e.g., very small particles dispersed in a liquid) that is susceptible to rapid dissolution, such as upon exposure to the higher pH environment of the intestine.

The term “matrix” or “film matrix” refers to the surroundings or medium constituting the film layer in which the active agent (e.g., Montelukast) is solubilized or distributed, and generally comprises a mixture of polymers and excipients. The film forming matrix supporting the API within the oral film dosage form can comprise about 40.0-99.0% dry w/w of the film layer.

Stability enhancing agents can be added to the film to prevent photodegradation, oxidation, and/or microbial contamination. Photodegradation inhibitors include ultraviolet radiation absorbers and pigments. Ultraviolet absorbers include hydroxyl benzophenones and hydroxyphenyl benzotriazoles. Pigments that can be added to the film include various metal oxides, such as titanium dioxide (TiO₂), ferric oxide (Fe₂O₃), iron oxide (Fe₃O₄), and zinc oxide (ZnO). In the cases of oral film dosage form the potential of photodegradation of the film dosage form may be mitigated by the use of individual pouches as the final packaging material. According to one embodiment, the pouches are made out of laminated material, comprising some aluminum or reflective foil material preventing photodegradation of the film and products contained therein. Microbial contamination may be controlled by the use of antimicrobial agent such as methyl, ethyl or propyl paraben, sodium benzoate, benzoic acid, sorbic acid, potassium sorbate, propionic acid or a combination of the above.

Other additives, such as excipients or adjuvants, can be incorporated into the film include flavors, sweeteners, coloring agents (e.g., dyes), plasticizers, and other conventional additives that do not deleteriously affect transmucosal delivery of the active agent, oral mucoadhesivity, or their important film properties.

The film can be used in a monolayer, bilayer or other multilayer form.

According to an embodiment, the bilayer film dosage form comprises a first layer having the API and a second layer having agents such as a taste-masking agent, backing agent for protecting the first layer, and/or a permeation enhancer. The second layer can also be used to favor the directed absorption through the oral mucosa (unidirectional absorption). Other embodiments could have the same API or a different API present in the second layer to enterally deliver the active with a controlled release profile. Alternatively, an active agent in the second layer could be used to modify the absorption of the active agent in the first layer.

A safe and effective amount generally refers to an amount that provides a beneficial or therapeutic effect, i.e., provides a curing or mitigating effect on disease or disease symptoms, but which is sufficiently low to avoid severe or life-threatening side effects when the active agent is administered and delivered transmucosally and/or enterally.

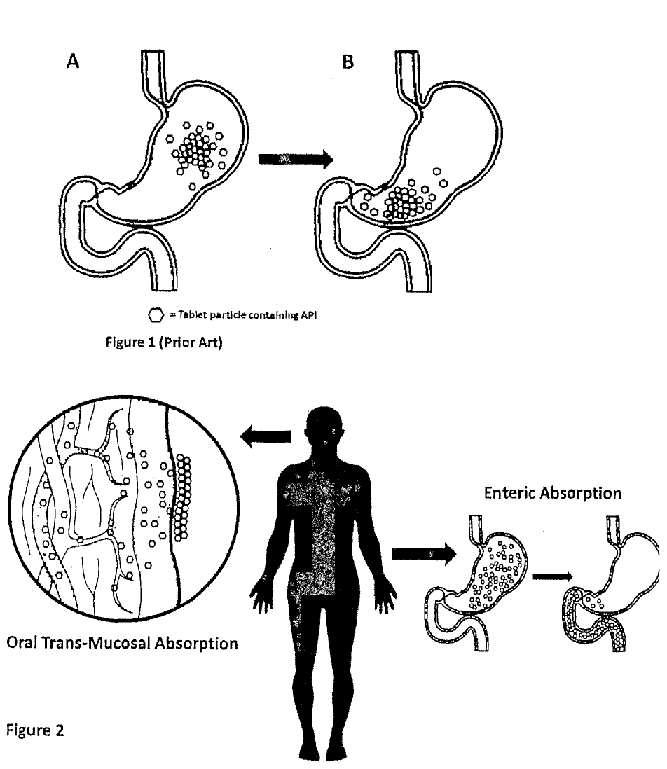

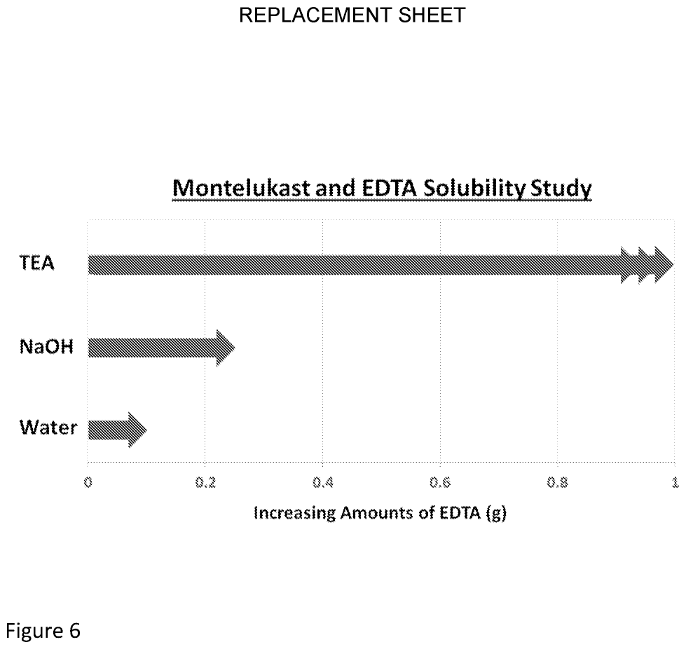

Montelukast solubility in aqueous media is dependent on the pH. It has been found that MTL exhibits increasing solubility at alkaline (basic) pH above 7.5 and is found to rapidly precipitate in media below pH 7.5. This has been experimentally shown by Okumu et al (Okumu, Pharm. Res, 25, 12, 2008), see FIG. 6, where MTL alone or in the presence of surfactants only displays a marked increase in solubility above pH 7.5. This study has also shown that although the impact of surfactants may marginally increase MTL solubility, it is only at alkaline (basic) pH environments that MTL readily solubilizes. Nevertheless, other parameters than solubility can influence the dissolution rate of the Montelukast i.e., the dosage form appears to have a significant impact on the dissolution rate. In certain embodiments the Montelukast is present as a dissolved form in the dosage form matrix which will dissolve and/or disintegrate in the mouth to allow the MTL to precipitate in saliva before being swallowed. Conversely, FIG. 1, is showing the schematic representation of the dissolution of an oral dosage form of MTL, such as a conventional tablet. FIG. 1A depicts the initial disintegration of the tablet in the stomach. FIG. 1B depicts the tablet disintegration after 10-15 minutes, where due to slower disintegration, the tablet pieces remain concentrated in a localized cluster limiting the dissolution and potential absorption. This limiting impediment is further exacerbated due to the poor solubility of MTL in acidic environments such as the stomach. Since MTL has an especially low solubility at low pH, the high concentration of MTL following disintegration of the tablet further increases the insolubility of MTL thereby potentially further reducing the bioavailability of the API.

In certain embodiments, the active agent can be distributed in the film matrix in the form of micro- or nano-particles.

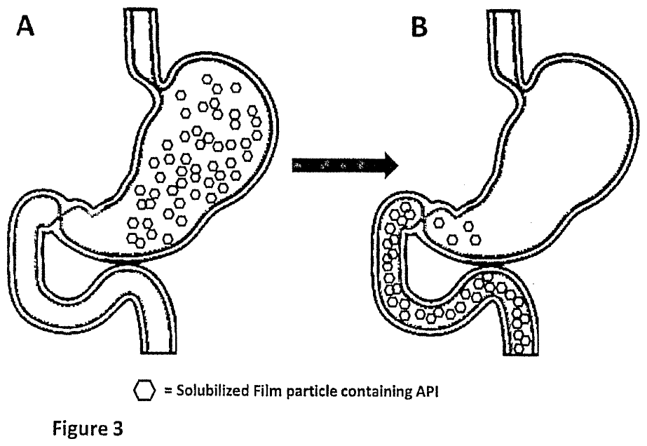

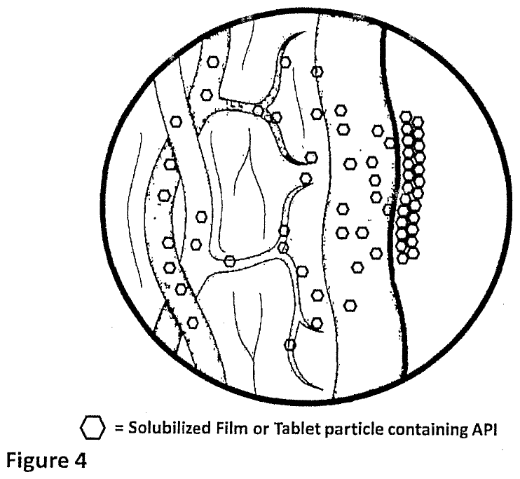

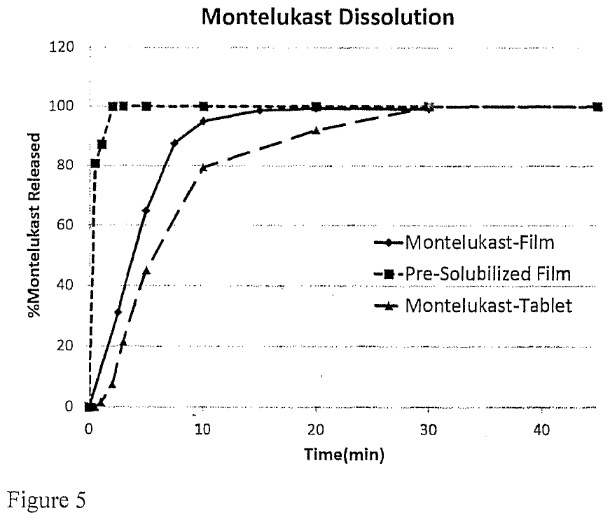

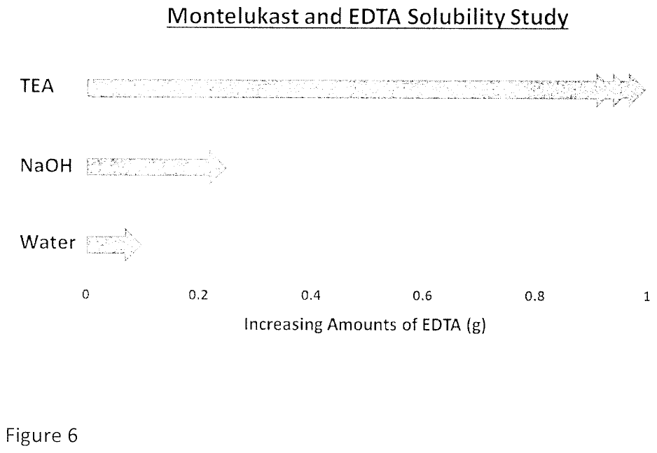

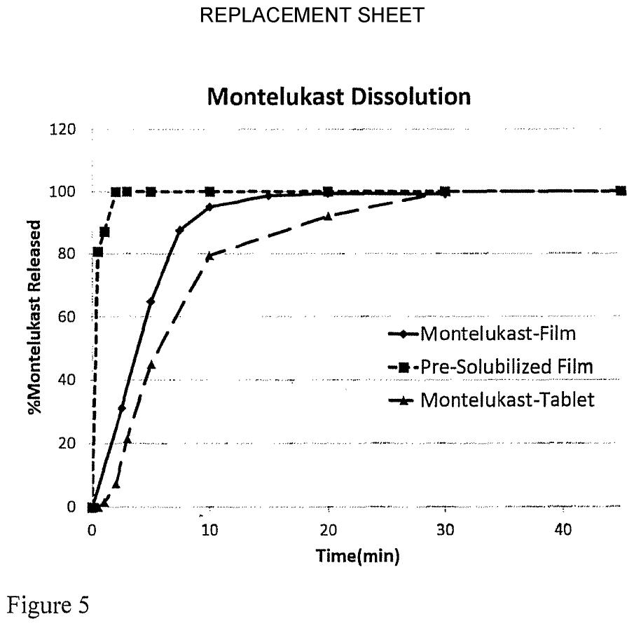

According to an aspect of the present disclosure, to mitigate the shortcomings of the abovementioned Montelukast tablet oral dosage form, it is herein disclosed a film oral dosage form wherein a leukotriene receptor antagonist (e.g., MTL) is administered via enteric absorption (FIG. 2B) alone or in combination with oral transmucosal and/or sublingual absorption (FIG. 2A) In certain embodiments, the film oral dosage form is designed to disintegrate in the mouth and allow a solubilized active agent to precipitate in the mouth and be swallowed, thereby delivering the API into the stomach as a fine precipitate suspended in aqueous medium. Referring now to FIG. 3A, upon reaching the stomach, the swallowed API precipitate in suspension is significantly more homogenously distributed throughout the stomach compared to the slowly disintegrating tablet dosage form which enters the stomach in relatively large solid particles or fragments containing the active agent. In this way, uptake of the film oral dosage form is believed to be less limited by the gastric emptying cycles. The lack of solid matrix retaining the API favors transport of the API throughout the stomach thus mitigating the effect of the low solubility of the API at low pH. As per the dissolution profile (FIG. 5) which clearly shows that once the dosage form is dissolved and/or disintegrated and the MTL precipitates in the saliva, its rate of dissolution in the intestine is much faster compared to that of the tablet. It is hypothesized that the precipitated MTL in saliva has a much smaller particle size and can escape the stomach via the pylorus as very fine particles suspended in a liquid, allowing for faster and higher absorption.

In certain embodiments, the film layer containing the active agent dissolves and/or disintegrates in the oral cavity upon contact with saliva. While the film dissolves and/or disintegrates, the Montelukast (or other leukotriene receptor antagonist) precipitates in the saliva (Montelukast API precipitates below pH 8) thus forming an API precipitate suspension in the saliva. The suspended API is then swallowed and reaches the stomach as a dispersed precipitate, improving the bioavailability of the Montelukast API. The pre-solubilized film at least mitigates the dissolution problem associated with the poor solubility of Montelukast in the patient's acidic stomach conditions. The poor solubility is generally amplified by the presence of a concentrated form of MTL. Though buccal and/or sublingual absorption may arise, the drug is predominantly absorbed enterally. As such, the oral film dosage can be used to overcome the solubility problem encountered when having Montelukast sodium present in the stomach in a solid or undissolved form. According to an embodiment of the disclosed oral dosage form, Montelukast film particulates reach the stomach already in a suspended/precipitated form, meaning that the Montelukast solubilized in the dosage form and precipitates in the oral cavity and/or esophagus, resulting in a suspended Montelukast precipitate being delivered to the stomach. As such, the pre-solubilized Montelukast in the dosage form has an improved bioavailability derived at least in part from the fact that the API is delivered to the stomach in a dispersed and thus less concentrated form than conventional tablets. The suspended precipitate thus exhibits an improvement in bioavailability when compared with tablets which must initially be dissolved in the stomach before being absorbed. The improved bioavailability can lead to increased transport of the active agent across the blood-brain barrier, allowing lower doses and/or more effective treatment. The administration of a Montelukast API suspension to the stomach at least mitigates solubility related problems arising in or with other Montelukast oral dosage forms such as swallowable and chewable tablets. Yet, according to an embodiment of the present invention, the administration of the suspended form through a film dosage form at least mitigates stability problems typically associated with API administered through liquid medium. In addition, the orally precipitated Montelukast is likely able to reach the small intestine quicker through the pylorus than other oral dosage forms of Montelukast or other Leukotriene receptor antagonist. According to an aspect of the present disclosure, using the preferred oral film dosage form, a dosage of up to a maximum of 20 mg a day of Montelukast is sufficient to alleviate symptoms or treat conditions associated with neuroinflammation. An essential element of such oral film dosage form is its ability to maintain Montelukast in a condition promoting its solubility, i.e. alkaline pH. According to some embodiments, the Montelukast oral film has an alkaline surface. The alkaline surface pH signifies that the film maintains Montelukast under alkaline conditions favoring its solubility and preventing recrystallization of the Montelukast. Recrystallization of the Montelukast is associated with unstable oral films. The Montelukast oral film preferably has a surface pH greater than pH 7.5, preferably greater than 8.0 and more preferably greater than 8.5.

Another embodiment of the oral dosage form comprises a capsule dosage form (e.g., a gelatin or cellulose—base capsule) containing the leukotriene inhibitor solubilized or distributed as an amorphous precipitate in a polymer matrix that disintegrates or dissolves in an aqueous medium. According to this dosage form, the oral dosage form of Montelukast is taken orally by the patient. Upon reaching the stomach the capsule shell is solubilized thus delivering the solubilized or amorphous precipitate of Montelukast (or other leukotriene receptor antagonist) into the aqueous medium of the stomach. Such precipitate will be rapidly distributed throughout the stomach and mitigates the shortcoming related to tablets and chewables. Since the active agent is already in a liquid medium in a solubilized or amorphous precipitate in suspension form, the oral capsule dosage form effectively mitigates low dosage bioavailability problems. Therefore, the Montelukast capsule allows the Montelukast to reach the stomach as a pre-solubilized, amorphous precipitate in suspension. It is possible that the stomach conditions, unfavorable to the dissolution of Montelukast tablets and chewables, result in some precipitation of the Montelukast in the stomach. However, since the Montelukast is already in a solubilized form or dispersed precipitate in aqueous medium, the extent of precipitation should be less than the loss of efficacy associated with the need to solubilize the Montelukast tablet in the stomach.

Leukotriene blockers or inhibitors (i.e., leukotriene receptor antagonists and leukotriene synthesis inhibitors) can function to improve cognitive impairment by reducing the neuroinflammatory response within the brain. Leukotriene blockers, such as MTL, must therefore cross the blood-brain barrier and accumulate in the CSF. Consequently, during clinical trials, patients were tested for CSF levels of MTL after 3 and 7 hours respectively, (see Table 1). What is most surprising about this finding is that between the 3- and 7-hour test points, the concentration of MTL continued to increase. This is particularly unexpected as the plasma levels show a Tmax value between 2-4 hours indicating that the maximum accumulated concentration is rapidly reached in the blood. The rapid accumulation of the Montelukast in the patients' blood is attributable to the enteral administration of Montelukast delivered to the stomach as an amorphous precipitate suspended in aqueous medium. As only two data points were taken during our clinical study it remains unclear if the time point at 7 hours represents the Cmax, or if the Cmax occurs after 7 hours as more MTL accumulates but is cleared at a much slower rate. This is of great significance when compared to the known treatments, wherein a strict regimen of continuous dosing was required to maintain effective levels of MTL for cognitive improvement. According to the present disclosure, a dosage of up to a daily maximum of about 20 mg or 25 mg is sufficient for treatment of the neuroinflammatory condition.

An effective concentration of Montelukast in the CSF is obtained via administration of the Montelukast according to the disclosed methods and dosage forms. Sufficient level of Montelukast is attained in the CSF because the Montelukast reaches the stomach in a pre-solubilized form thereby enhancing the absorption and bioavailability of the Montelukast. Accordingly, a disclosed method of treating neurodegenerative or neuroinflammatory disorder comprises the step of enterally delivering to a person or other animal in need of treatment for a neurodegenerative disease or neuroinflammatory disorder via a film dosage form, a safe and effective amount of solubilized Montelukast.

Our data clearly demonstrates that regular dosing of Montelukast oral film every 2 hours is not necessary to maintain effective levels of MTL in the CSF. The higher bioavailability of the Montelukast in the CSF is due to the administration of Montelukast in such a dosage that the active agent is delivered to the stomach as a suspended precipitate in aqueous medium, thus mitigating the hurdles related to solubilizing Montelukast swallowable tablets or chewable tablets in the stomach. Accordingly, administering Montelukast under a liquid dosage form or a dosage form for buccal dissolution wherein the API precipitates in the saliva and yields an API suspension in saliva, increases the bioavailability of Montelukast in the subjects. It is thus desired to administer a form of Montelukast (or other leukotriene receptor antagonist) orally for dissolution or disintegration of the oral dosage form before reaching the stomach.

TABLE 1
Pharmacokinetic Data for CSF Concentrations

| | Concentration | Concentration |
|---|---|---|
| | at 3 hours | at 7 hours |
| Sample | (ng/ml) | (ng/ml) |
| MTL03 Film | 3.60 | 4.20 |

We have performed a clinical study of our product to determine the pharmacokinetics of the API loaded into this pharmaceutical platform. Our film product and the Singulair® product both contain 10 mg MTL free base. Singulair® is the marketed formulation of MTL, commonly prescribed for asthma sufferers. It consists of a 10 mg loaded API tablet. The Cmax and Tmax values are listed below, see Table 2. Results indicate that we have approximately 1.5 times the Cmax and AUC values compared to the Singulair® reference. These higher values for our films means that we could load less API into the film product and achieve the same Cmax/AUC as the Singulair® reference product. The major difference between the disclosed Intelgenx prototype and Singulair® is the physical state of the MTL once it reaches the stomach. In the Singulair® product, the MTL reaches the stomach in a compressed solid state and thus must solubilize in the stomach under unfavorable conditions. In contrast, the disclosed MTL03 oral film dosage form comprises solubilized MTL, which is placed in the mouth and allowed to dissolve before being swallowed. Upon dissolution of the disclosed MTL03 oral film dosage form, the MTL precipitated in the oral cavity while the remainder of the dosage form disintegrated and/or dissolved, creating a MTL precipitate ultimately suspended in aqueous medium (i.e. saliva). As such, the MTL contained within the disclosed MTL03 prototype reaches the stomach in an already pre-solubilized state, meaning that the matrix has been dissolved or disintegrated, exposing the MTL precipitate to the stomach fluid. The MTL is then transferred to the small intestine via the pylori. Since the MTL is already present as a suspended precipitate, the MTL may more easily reach the small intestine through the leaking pylori. It is well known that the pylori is not leak proof and allows some liquid to flow through even in its closed position. As such, MTL of the disclosed MTL03 film dosage once in the stomach is believed to more easily traverse the pylori. Administering MTL enterally as a suspended precipitate in aqueous medium improves bioavailability. To further support the fact that MTL exhibits increased bioavailability when administered through such dosage form, we compared the pharmacokinetics of MTL provided to the FDA under New Drug Application (NDA) 020830 (see Table 2). Table 2 shows that the chewable oral dosage form is more readily bioavailable than the solid dosage. The chewable MTL dosage is partly solubilized, hence its bioavailability is superior to the tablet bioavailability. In particular, the chewable dosage form is about 1.17 times more bioavailable than the tablet when administered in a fasting subject (compared using available bioavailability date shown in Table 2). Therefore, through comparative extrapolation, the disclosed MTL03 film dosage which contains MTL solubilized in the film matrix and precipitates in the saliva once the matrix dissolves has proven to be 1.5 times more bioavailable than the tablet when comparing the area under the curve (AUC) (see Tables 3 & 4). It is believed that administering the MTL as a precipitate suspension that is free from the film or tablet matrix improves the bioavailability of the MTL when compared with the corresponding tablet and chewable. This improved bioavailability is believed to be at least in part caused by the increased contact area of the precipitate API. In addition, the MTL is delivered in the stomach in a less concentrated manner than corresponding tablet and chewable oral dosage forms (see FIGS. 1 and 3).

TABLE 2
Bioavailability comparison between the tablet
and the chewable oral dosage forms

| Dosage form | Bioavailability |
|---|---|
| Singulair ® Film coated tablet | 64% (not affected by food) |
| Chewable | 75% (fasted) 63% (with food) |

According to an embodiment, the method for treating a neurodegenerative disease or neuroinflammatory disorder, comprises the step of (a) enterally delivering to a person or other animal in need of treatment for a neurodegenerative disease or neuroinflammatory disorder via a film dosage form, a safe and effective amount of Montelukast. Preferably, the Montelukast is orally administered via an oral film dosage comprising MTL or any other suitable salt, ester or prodrug thereof. According to the present method of treatment, the Montelukast is at least substantially solubilized in the film dosage form and administered orally with a film matrix that dissolves and/or disintegrates in contact with aqueous medium such as saliva when in the oral cavity. The MTL precipitates upon dissolution of the film matrix in the saliva in the persons or animals oral cavity. Furthermore, the pharmacokinetic data for the disclosed MTL03 MTL dosage form show that absorption is significantly higher than for the branded form Montelukast Singulair® product (tablet). Therefore administering MTL as a film dosage form having a matrix that rapidly dissolves or disintegrates (i.e. that dissolved or disintegrates within less than 10 minutes, preferably between 2 and 7 minutes and more preferably within 3 to 5 minutes) to yield a precipitate suspension in aqueous medium before reaching the stomach, markedly improves the bioavailability of Montelukast, as compared to oral tablets or capsules where Montelukast is held in a dosage form matrix that is resistant to solubilization and absorption of the active agent. According to a preferred aspect of the present disclosure, a leukotriene receptor antagonist, such as Montelukast, is solubilized in the oral film dosage form.

According to another aspect of the present disclosure, the leukotriene receptor antagonist is present in the film as a particulate active in an oral film dosage form. In such an alternate embodiment of the disclosure, this particulate API is held in the oral film matrix, in which the film matrix will dissolve and/or disintegrate when in contact with an aqueous medium (i.e. saliva). Upon dissolution and/or disintegration of the film matrix, the particulate API will be present as a particulate suspension in aqueous medium. The particulate API is preferably in amorphous form in the film matrix.

TABLE 3
Pharmacokinetic Data for Plasma Concentrations

| Sample | Cmax (ng/ml) | Tmax (hrs) | AUC |
|---|---|---|---|
| MTL03 Film | 599 | 2.70 | 3910 |
| (alkaline) | | | |
| Singulair ® | 386 | 3.63 | 2617 |
| product | | | |
| Ratio alkaline | 1.55 | 0.74 | 1.49 |
| Film/Tablet | | | |

TABLE 4
Comparison of bioavailability between different dosage forms

| Dosage form | Bioavailability |
|---|---|
| Singulair ® tablet product | 1 |
| MTL03 Film | 1.5 |

Once administered, the oral film is preferably applied against the subjects' oral mucosa where it will be adhered to and enter in contact with the subject's saliva. Contact between the film and the saliva dissolves and/or disintegrates the film in the oral cavity. The dissolved and/or disintegrated oral film matrix advantageously allows precipitation of the active agent in the oral cavity of a subject. The precipitate is swallowed for enteral administration as a suspended precipitate in aqueous medium.

A preferred amount of MTL per unit dosage form is from about 0.5 mg to about 25 mg, preferably about 1 mg to about 25 mg, more preferably about 5 mg to about 10 mg.

Illustrative, but non-limiting, examples of formulations used to prepare a MTL oral films is shown in Tables 5-11.

TABLE 5
MTL01

| | | | Composition | Composition |
|---|---|---|---|---|
| Item # | Description | Function | (% wet w/w) | % dry (w/w) |
| A | Methanol | Solvent (will be removed | 0.29 | — |
| | | during manufacturing) | | |
| B | Purified Water | Solvent (will be removed | 79.68 | — |
| | | during manufacturing) | | |
| 1 | Starch | Filler | 1.81 | 9.04 |
| 2 | HPC SL | Film former polymer | 8.37 | 41.79 |
| 3 | Xanthan gum | Thickener | 0.88 | 4.39 |
| 4 | Sucralose | Sweetener | 0.44 | 2.20 |
| 5 | Glycerol | Plasticizer | 1.85 | 9.24 |
| 6 | Montelukast | Active | 3.3 | 16.48 |
| | Sodium | | | |
| 7 | Ascorbic acid | Stabilizer | 0.01 | 0.05 |
| 8 | Methylparaben | Anti-microbial agent | 0.11 | 0.55 |
| 9 | Titanium Dioxide | Opacifier | 0.27 | 1.35 |
| 10 | Yellow #10 | Color | 0.28 | 1.40 |
| 11 | HPC LF | Film former polymer | 0.73 | 3.64 |
| 12 | Calcium | pH Modifier | 0.51 | 2.55 |
| | Carbonate | | | |
| 13 | Sodium | Permeation Enhancer | 1.47 | 7.34 |
| | glycocholate | | | |
| Total | | | 100 | 100.00 |

TABLE 6
MTL02

| | | | Composition | Composition |
|---|---|---|---|---|
| Item # | Description | Function | (% wet w/w) | % dry (w/w) |
| A | Methanol | Solvent (will be removed | 0.2 | — |
| | | during manufacturing) | | |
| B | Purified Water | Solvent (will be removed | 79.68 | — |
| | | during manufacturing) | | |
| 1 | Povidone | Film former polymer | 11.08 | 55.07 |
| 2 | Locust Bean Gum | Thickener | 0.88 | 4.37 |
| 3 | PEG 300 | Plasticizer | 0.15 | 0.75 |
| 4 | Labrafil | Permeation Enhancer | 0.89 | 4.42 |
| | M1944CS | | | |
| 5 | Sucralose | Sweetener | 0.44 | 2.19 |
| 6 | Citric Acid | pH Modifier | 0.61 | 3.03 |
| 7 | Montelukast | Active | 3.3 | 16.40 |
| | Sodium | | | |
| 8 | Sodium Edetate | Stabilizer | 0.01 | 0.05 |
| 9 | Propylparaben | Anti-microbial agent | 0.1 | 0.50 |
| 10 | Titanium Dioxide | Opacifier | 0.27 | 1.34 |
| 11 | Yellow #10 | Color | 0.28 | 1.39 |
| 12 | HPC-GXF | Film former polymer | 2.11 | 10.49 |
| Total | | | 100.00 | 100.00 |

TABLE 7
MTL03

| | | | Composition | Composition |
|---|---|---|---|---|
| Item # | Description | Function | (% wet (w/w) | % dry (w/w) |
| A | Methanol | Solvent (will be removed | 0.29 | — |
| | | during manufacturing) | | |
| B | Purified Water | Solvent (will be removed | 79.68 | — |
| | | during manufacturing) | | |
| 1 | Starch | Filler | 1.81 | 9.04 |
| 2 | Pullulan | Film former polymer | 8.37 | 41.79 |
| 3 | Tara gum | Viscosity Modifier | 0.88 | 4.39 |
| 4 | PEG 300 | Plasticizer | 0.15 | 0.75 |
| 5 | Sorbitol P60W | Plasticizer | 1.83 | 9.14 |
| 6 | Sucralose | Sweetener | 0.44 | 2.20 |
| 7 | Glycerol | Plasticizer | 1.85 | 9.24 |
| 8 | Montelukast | Active | 3.30 | 16.48 |
| | Sodium | | | |
| 9 | BHT | Stabilizer | 0.01 | 0.05 |
| 10 | Propylparaben | Anti-microbial agent | 0.11 | 0.55 |
| 11 | Titanium Dioxide | Opacifier | 0.27 | 1.35 |
| 12 | Yellow #10 | Color | 0.28 | 1.40 |
| 13 | HPC LF | Film former polymer | 0.73 | 3.64 |
| Total | | | 100.00 | 100.00 |

TABLE 8
MTL04

| | | | Composition | Composition |
|---|---|---|---|---|
| Item # | Description | Function | (% wet (w/w) | % dry (w/w) |
| A | Methanol | Solvent (will be removed | 0.29 | — |
| | | during manufacturing) | | |
| B | Purified Water | Solvent (will be removed | 79.68 | — |
| | | during manufacturing) | | |
| 1 | Starch | Filler | 0.74 | 3.69 |
| 2 | PEO 200K | Film former polymer | 8.37 | 41.79 |
| 3 | PEO 100K | Film former polymer | 2.35 | 11.73 |
| 4 | Menthol | Flavor | 1.3 | 6.49 |
| 5 | Sorbitol P60W | Plasticizer | 1.68 | 8.39 |
| 6 | Sucralose | Sweetener | 0.44 | 2.20 |
| 7 | Citric Acid | pH Modifier | 0.45 | 2.25 |
| 8 | Montelukast | Active | 3.3 | 16.48 |
| | Sodium | | | |
| 9 | Sodium Sulfite | Stabilizer | 0.01 | 0.05 |
| 10 | Methylparaben | Anti-microbial agent | 0.11 | 0.55 |
| 11 | Titanium Dioxide | Opacifier | 0.27 | 1.35 |
| 12 | Yellow #10 | Color | 0.28 | 1.40 |
| 13 | HPC JF | Film former polymer | 0.73 | 3.64 |
| Total | | | 100.00 | 100.00 |

TABLE 9
MTL05

| | | | Composition | Composition |
|---|---|---|---|---|
| Item # | Description | Function | (% wet (w/w) | % dry (w/w) |
| A | Methanol | Solvent (will be removed | 0.29 | — |
| | | during manufacturing) | | |
| B | Purified Water | Solvent (will be removed | 78.66 | — |
| | | during manufacturing) | | |
| 1 | Sodium | pH Modifier | 0.65 | 3.09 |
| | Hydroxide | | | |
| 2 | HPMC E5 | Film former polymer | 3.21 | 15.25 |
| 3 | HPC-L | Film former polymer | 9.63 | 45.75 |
| 4 | PEG 300 | Plasticizer | 0.15 | 0.71 |
| 5 | Sorbitol P60W | Plasticizer | 1.83 | 8.69 |
| 6 | Sucralose | Sweetener | 0.44 | 2.09 |
| 7 | Sodium | Stabilizer | 0.59 | 2.80 |
| | Metabisulfite | | | |
| 8 | Montelukast | Active | 3.3 | 15.68 |
| | Sodium | | | |
| 9 | Sodium Edetate | Stabilizer | 0.01 | 0.05 |
| 10 | Propylparaben | Anti-microbial agent | 0.11 | 0.52 |
| 11 | Yellow #10 | Color | 0.28 | 1.33 |
| 12 | Oleic acid | Permeation Enhancer | 0.85 | 4.04 |
| Total | | | 100.00 | 100.00 |

TABLE 10
MTL06

| | | | Compo- | Compo- |
|---|---|---|---|---|
| | | | sition | sition |
| Item | | | (% wet | % dry |
| # | Description | Function | (w/w) | (w/w) |
| A | Methanol | Solvent (will be removed | 0.29 | — |
| | | during manufacturing) | | |
| B | Purified Water | Solvent (will be removed | 79.68 | — |
| | | during manufacturing) | | |
| 1 | Sodium | pH Modifier | 0.84 | 4.19 |
| | Hydroxide | | | |
| 2 | Pullulan | Film former polymer | 9.34 | 46.63 |
| 3 | Xanthan gum | Thickener | 1.88 | 9.39 |
| 4 | PEG 300 | Plasticizer | 0.15 | 0.75 |
| 5 | Sodium sulfite | Stabilizer | 0.65 | 3.25 |
| 6 | Sucralose | Sweetener | 0.44 | 2.20 |
| 7 | Glycerol | Plasticizer | 1.85 | 9.24 |
| 8 | Montelukast | Active | 3.3 | 16.48 |
| | Sodium | | | |
| 9 | Azone | Permeation Enhancer | 0.92 | 4.59 |
| 10 | Propylparaben | Anti-microbial agent | 0.11 | 0.55 |
| 11 | Titanium Dioxide | Opacifier | 0.27 | 1.35 |
| 12 | Yellow #10 | Color | 0.28 | 1.40 |
| Total | | | 100.00 | 100.00 |

TABLE 11
MTL07

| | | | Compo- | Compo- |
|---|---|---|---|---|
| | | | sition | sition |
| Item | | | % wet | % dry |
| # | Description | Function | (w/w) | (w/w) |
| A | Methanol | Solvent (will be removed | 0.29 | — |
| | | during manufacturing) | | |
| B | Purified Water | Solvent (will be removed | 79.68 | — |
| | | during manufacturing) | | |
| 1 | Ascorbic acid | Stabilizer | 0.97 | 4.84 |
| 2 | HPC-SL | Film former polymer | 9.66 | 48.23 |
| 3 | Xanthan gum | Thickener | 1.43 | 7.14 |
| 4 | PEG 300 | Plasticizer | 0.15 | 0.75 |
| 5 | Sorbitol P60W | Plasticizer | 1.83 | 9.14 |
| 6 | Sucralose | Sweetener | 0.44 | 2.20 |
| 7 | Labrafil | Permeation Enhancer | 1.02 | 5.09 |
| | M1944CS | | | |
| 8 | Montelukast | Active | 3.3 | 16.48 |
| | Sodium | | | |
| 9 | Sodium | Stabilizer | 0.84 | 4.19 |
| | metabisulfite | | | |
| 10 | Propylparaben | Anti-microbial agent | 0.11 | 0.55 |
| 11 | Yellow #10 | Color | 0.28 | 1.40 |
| Total | | | 100.00 | 100.00 |

Preparation of a film product typically involves casting or otherwise thinly spreading the liquid film formulation on a substrate, drying (e.g., evaporating) all or most of the solvent(s) from the film to produce a thin, solid film sheet of material, and cutting the solid film sheet into individual unit dosage forms.

FIG. 5 shows an increased rate of dissolution of the present film oral dosage form of MTL when compared with the Singulair® MTL tablet. In addition, disclosed in FIG. 5 is the dissolution of the present film oral dosage form taking into account the buccal delivery method. In these experiments the “pre-dissolved film” refers to a film that is pretreated to simulate conditions typical of when the film is applied to oral mucosa of a human subject. Under such simulated conditions, the film slowly disintegrates before being subjected to the dissolution experiment. This method is used for a more representative comparison of the swallowed tablet behavior in the stomach with that of the swallowed film; the film is again much faster. In general, the dissolutions were conducted under the following conditions. The dosage consists of a 10 mg unit of either film or tablet. A USP dissolution apparatus was used to measure the API release profiles. Each dissolution container was filled with 900 mL of phosphate based simulated saliva buffer pH 6.8. The paddle speed was set to 50 rpm and the temperature was kept at 37° C. Each pull point consisted of 8 mL and the time points were taken at 1, 2.5, 5, 7.5, 10, 15, 20, 30, 45. Samples were analyzed using UV absorption at 273 nm. Pre-solubilized Montelukast-Film dissolution was prepared by mixing a single film unit in 2 mL of simulated saliva buffer. This volume is considered to be representative of the volume of saliva generally found in the oral cavity under normal conditions. Data is summarized in Table 12.

TABLE 12

| | Time to 80% API released |
|---|---|
| Sample | (min.) |
| Montelukast-Films MTL03 & MTL10 | 6 |
| Pre-dissolved Montelukast-Films | 1 |
| Montelukast-Tablet | 10 |

It was found that the MTL03 and MTL10-films reached 80% API released after approximately 6 minutes, while for the MTL-tablet to reach the same level of released API required 10 minutes. This highlights the rapid disintegration advantage of the film based platform. However, the most significant improvement using our film technology is observed when comparing the tablet to the pre-solubilized MTL03 and MTL10-films. This experiment is particularly interesting as it is a more representative comparison of how API is released from swallowed MTL-tablets versus swallowed MTL03 and MTL10-films in the comparable environmental conditions. Surprisingly, the pre-dissolved MTL03 and MTL10-films reaches 80% released API in only approximately 1 minute. This clearly demonstrates how the MTL03 and MTL10-films platform releases MTL significantly more quickly than the MTL-tablet dosage. This is believed to contribute towards the observed improved bioavailability during our Phase I Clinical study.

As demonstrated above, the oral film of MTL (principally MTL03) exhibits improved bioavailability compared to presently marketed products available as tablets/granules or suspensions. It is believed that the increased bioavailability of the MTL is related to the state of the MTL within the oral film. According to some embodiments, improved bioavailability of the oral film dosage form critically linked to the incorporation of solubilized MTL, into the alkaline oral films, ensuring a rapid release of pre-solubilized therapeutic which is easily absorbed in the oral cavity and enterically. The alkalinity of the oral film as measured by the surface pH of the film favors dissolution of the MTL within the film. It is believed that the MTL remains soluble to some extent within the film due in part by the presence of residual solvent. Our preliminary results from manufacturing processes demonstrate the presence of between 5 to 9% dry w/w of residual solvent. As such, alkaline surface pH oral films of MTL (MTL01, MTL03, MTL05, MTL06 and MTL 07) are expected to exhibit the observed increased bioavailability of MTL03. The alkaline film layer is designed to keep MTL in a favorable solubilized condition that readily forms amorphous precipitates in the saliva upon oral administration of the film.

A significant challenge regarding Montelukast oral film formulations therefore pertains to the stability of the solubilized API during the manufacturing, processing and long term storage. Although solubilizing API significantly improves the bioavailability of the drug, it also potentially accelerates the degradation/decomposition pathways of the API leading to unwanted impurities. The present disclosure addresses why achieving a stable solubilized MTL product is unexpectedly challenging for those skilled in the art, and the process by which it can be accomplished using by using specific critical excipient to API ratios and mixing conditions.

Montelukast is known to degrade over time (M. M. Al Omani et al.) in a solid or liquid state when exposed to light, moisture or heat, yielding degradation products such as Montelukast sulfoxide (SO) and Montelukast cis-isomer {Journal of Pharmaceutical and Biomedical Analysis, 45, 2007, 465-471}. Singulair® chewable tablets exposed to sunlight, show an increased amount of the Montelukast sulfoxide impurity of 2.4% after 3 weeks. Furthermore, Montelukast in 0.1 M hydrochloric acid solution exposed to a sodium vapor lamp for 6 hours, leads to a 14.6% increase in the amount of Montelukast m-isomer.

Accordingly, because maintaining solubilized MTL during the formulation, production and processing is necessary for ensuring consistent bioavailability after prolonged storage of the film dosage form, the choice of stabilizer or antioxidant can be important. The choice of antioxidant/stabilizer is limited to molecules which will not lead to, or interact with, the API in such a way as to cause precipitation. This challenge would not be encountered in tablet formulations, as MTL, is kept and used in its solid state. Solubilized MTL is particularly sensitive to changes in the pH environment and precipitates at lower pH, such as below 8. Solubilized MTL is also negatively charged which can lead to unwanted complexations. Therefore, the choice and amount of antioxidant is further limited and excludes highly acidic molecules or molecules which may associate with the API covalently or non-covalently to form insoluble precipitate complexes and/or aggregated material.

Experimental studies have revealed that MTL is particularly susceptible when in its solubilized state to metal catalyzed degradation as well as other oxidative or photo-induced decomposition pathways. Existing MTL dosage forms are predominantly found as tablets, tablet variants or suspensions in which MTL is a solid or a suspension. In these formulas antioxidants/stabilizers can be directly added as solid material or applied to the product indirectly (spray coatings, shells or film coating). There is no need to consider antioxidant/stabilizer interactions which would precipitate MTL in a tablet dosage form, as it is already a solid.

Our studies have shown that film formulas of MTL using only BHT as an antioxidant, exhibit increased impurities after 3 months in the stability chamber (25° C./65% RH). We have therefore investigated the use of chelating agents to prevent the observed extent of degradation. Examples of chelating agent include but is not limited to, molecules such as disodium edetate (EDTA), tetra sodium edetate, calcium disodium edetate, pentetic acid (DTPA), citric acid (CA), DL-2,3-Dimercapto-1-propanesulfonic (DMPS), dimercaptosuccinic acid (DMSA), monoisoamyl DMSA (MiADMSA) alpha lipoic acid (ALA), glutathione, N-acetyl-cystein (NAC), vitamin C, (2)-2-amino-3-methyl-3-sulfanylbutanoic acid, dithioglycerine, N-(alpha-L-arabinofuranos-1-yl)-L-cystein or nitrilotriacetic acid (NTP). In some cases, chelators such as EDTA are offered as different salts which exhibit more alkaline pH effects on the aqueous media, however these molecules, such as tetra sodium edetate or disodium calcium edetate do not perform as well in maintaining MTL stability in long term studies.

It is known that chelators such as EDTA are highly effective at sequestering the metal ions responsible for catalyzing the sulphoxide impurity formation. The greater the concentration of EDTA the greater the stability of the MTL API. However, addition of chelators in an aqueous medium in general leads to deprotonation of the chelators and consequent acidification of the aqueous blend. This is problematic as MTL solubility is particularly sensitive to changes in the pH of the environment and rapidly precipitates at pH below 8. In fact as seen in FIG. 1 below only a limited amount of EDTA can be added to a solution of MTL before precipitation is observed.

TABLE 13
EDTA concentration is increased while MTL and water amounts
are kept constant, EDTA concentration (w/w dry).

| | Low | | | | | High |
|---|---|---|---|---|---|---|
| % EDTA | 0% | 0.427% | 0.855% | 1.711% | 2.832% | 4.542% |
| MTL | YES | YES | YES | YES | NO | NO |
| Solubility | | | | | | |

The solubility and stability of MTL are critical parameters to consider when formulating oral films that will generate a reproducible target bioavailability and stable product. Therefore, optimal formulations of MTL will need to balance the amount of API with EDTA in order to achieve the needed stability while maintaining a solubilized drug component. This can be achieved using several strategies: (1) balancing the ratio of EDTA to MTL (MTL itself is a basifying agent), (2) using base modifying excipients to compensate for increasing amounts of EDTA, and (3) application of alkaline buffering components.

The following experiments will provide detailed information regarding the ratios in which these excipients can be combined to achieve the needed API stability while maintaining a solubilized API.

MTL Stability with Increasing EDTA

Results for the stability of MTL with increasing EDTA concentration up to 2 weeks at 50° C. have been analyzed. In general, the SO impurity is observed to increase over time while the Cis impurity is more stable. We have tracked the SO impurity as an indicator of the efficacy of EDTA to prevent its formation, see the Table 1 below. In general the greater the amount of EDTA the less SO impurities are formed. Overall, these results indicate that using 1.6% EDTA we keep the total SO impurities below 0.5%.

TABLE 14
Stability Results of MTL with Increasing EDTA Concentrations:

| | Total SO | Total SO | Total SO |
|---|---|---|---|
| Prototype | Impurities: W0 | Impurities: W1 | Impurities: W2 |
| 0% dry w/w EDTA | 0.19 | 0.59 | 1.26 |
| 0.4% dry w/w EDTA | 0.17 | 0.43 | 0.69 |
| 0.8% dry w/w EDTA | 0.43 | 0.36 | 0.58 |
| 1.6% dry w/w EDTA | 0.23 | 0.43 | 0.48 |
| 2.5% dry w/w EDTA | 0.12 | 0.35 | 0.44 |
| 3.3% dry w/w EDTA | 0.11 | 0.17 | 0.20 |

Holding Study of MTL Solubility in the Presence of EDTA

A second surprising challenge for using EDTA to stabilize MTL, is that regardless of the concentration of EDTA, overtime nearly 100% of the EDTA is observed to precipitate. Higher concentrations of ETDA lead to accelerated precipitation of MTL within minutes, while lower concentrations result in precipitation only after 10 days. This is of particular importance as it means that the holding time of the blend should never be longer than the observed time to precipitation. These are binary mixtures in water, the blend behavior is likely to be different (yet similar) in a blend with higher viscosity and many more excipients. This is important for the wet blend holding time during manufacturing.

TABLE 15
MTL Supernatant Concentration and Amount Precipitated:

| | | Supernatant | | | | |
|---|---|---|---|---|---|---|
| | EDTA | Conc. | % | % | Time to | |
| Sample | (g) | (mg/ml) | Dissolved | Precipitated | precipitate | |
| 1 | 0.035 | 0.642 | 1.60 | 98.4 | 10 | days |
| 2 | 0.070 | 0.536 | 1.33 | 98.67 | 7 | days |
| 3 | 0.150 | 0.494 | 1.23 | 98.77 | 7 | days |
| 4 | 0.225 | 0.494 | 1.23 | 98.77 | 1 | hour |

| | | | | | |
|---|---|---|---|---|---|
| 5 | 0.400 | 0 | 0 | 100 | instantly |

*MTL was kept constant at 1.125 g

### MTL Solubility and EDTA: Application of Basifying Agents

Basifying agents (i.e., additives that cause pH to increase) have been examined to determine if their addition can be used to maintain solubilized MTL while increasing the level of EDTA to obtain improved stability. In these experiments we have used both ionic salts and organic bases to basify the blend.

TABLE 16

| | | Basifying | | | |
|---|---|---|---|---|---|
| | MTL | Agent | EDTA | | |
| Sample | (g) | (g) | (g) | pH | Solubility of MTL |
| Control A | 1.125 | none | 0.087 | 8 | Solubilize, clear |
| | | | | | yellow |
| Control B | 1.125 | none | 0.150 | 8-9 | Solubilize, clear |
| | | | | | yellow |
| Control C | 1.125 | none | 0.225 | 8-9 | Precipitates MTL |
| 1 | 1.125 | NaOH, 1 g of | 0.225 | 10 | Solubilize, clear |
| | | 1M soln. | | | yellow |
| 2 | 1.125 | NaOH, 1 g of | 0.300 | 10 | Precipitates MTL |
| | | 1M soln. | | | |
| 3 | 1.125 | NaOH, 3 g of | 0.300 | 12 | Precipitates MTL, |
| | | 1M soln. | | | creamy blend |
| 4 | 1.125 | 0.100 g TEA | 0.225 | 9 | Solubilize, clear |
| | | pure | | | very faint yellow |
| 5 | 1.125 | 0.200 g TEA | 0.500 | 10 | Solubilize, clear |
| | | pure | | | very faint yellow |
| 6 | 1.125 | 0.400 g TEA | 1.000 | 10.5 | Solubilize, clear |
| | | pure | | | very faint yellow |

Comparing the Controls A and B demonstrates the threshold for the maximum amount of EDTA that can be added to the solution while maintaining MTL solubility. Adding a portion of NaOH basifying agent to these mixtures allows more EDTA to be added while maintaining solubilized MTL. However, this ratio does not scale linearly. For example, 1 g of a 1M NaOH is sufficient to solubilize MTL in the presence of 0.225 g EDTA, however if we increase the EDTA to 0.300 g, even tripling the amount of NaOH does not solubilize the MTL. These blends containing increasing amounts of NaOH in the presence of precipitated MTL, exhibit a surprising increase in viscosity and texture, generating a white cream-like blend; likely indicative of problematic excipient to API interactions in highly alkaline salt environments. Similar experiments have been performed using an organic base triethyl amine (TEA). Surprisingly when using a solubilized organic base the ratio of TEA to EDTA is directly scalable; as we add more TEA, we are able to add proportionally more EDTA, while maintaining solubilized MTL. Addition of TEA to the aqueous blend likely leads to its conversion into the corresponding ammonium salt, therefore direct addition of ammonium as a basifying agent will generate the same result. Another surprising aspect of using the TEA is that although MTL is solubilized, the color of the solubilized API is significantly different. When using the TEA the blend is actually only very faintly yellow compared to the normally bright clear yellow solution we observe when employing NaOH. Samples using both NaOH and TEA to maintain MTL solubility have been analyzed to determine if the color change is linked to MTL degradation and impurity formation. It was found that there was not a significant difference between the impurities for these two samples, in fact faintly yellow solution showed fewer impurities than the bright yellow blend with NaOH. See FIG. 6, which summarizes this series of experiments. Accordingly, stabilization of the Montelukast film using EDTA is preferred using a liquid or water soluble weak organic base such as TEA.

FIG. 6 is a graphical representation of the solubility limits of MTL in solutions containing EDTA. In these experiments the amount of MTL and water used are kept constant and are proportional to what is found in the formula. Increasing amounts of basifying agents (NaOH and TEA) and EDTA are used and we visually monitor the precipitation of MTL. The arrows for each sample listed terminate when MTL precipitates from solution. It was found that in a solution containing only water and MTL, we can add up to 0.15 g of EDTA after which the MTL begins to precipitate due to acidification from the EDTA. Adding the basifying agents allows more EDTA to be added to the solution without precipitating MTL; the greatest increase in amount of EDTA added was found using TEA.

A third possibility for basifying the blend to compensate for the acidity of EDTA and other chelators, is to actually increase the amount of dissolved MTL. MTL itself contributes significantly to the basification of the solution and is freely soluble in pure water. In the absence of other basifying agents, MTL is responsible for the needed basification/buffering of the blend at alkaline pH to allow incorporation of the minimum amount of EDTA required for stability. However, the addition of too much MTL unexpectedly has a significant negative impact on the film mechanical properties and blending. As MTL % w/w increases, the film becomes increasingly brittle and sticky, leading to strong liner interactions, which prevents easy release of the product during packaging steps. The range and ratio of MTL with respect to EDTA is critical from this second perspective, so as to not generate a product with poor flexibility, mechanical strength and liner release, which will impede scaled up manufacturing. MTL also behaves as an amphiphilic molecule in solution, acting to stabilize bubbles and foam in the blend during mixing when present at high relative concentrations. This will slow down manufacturing as longer degassing conditions will need to be applied. The ratio of MTL to EDTA is therefore quite sensitive for the development of a functional pharmaceutical product. Accordingly, stabilization of the Montelukast film using EDTA is preferred using a liquid or water soluble weak organic base such as TEA.

According to some embodiment of the disclosed oral film dosage form, the film layer comprises between 0.01 to 0.04% dry w/w of BHT with between 1.6 to 2.0% dry w/w of EDTA (disodium edetate)

### Ratio of MTL to EDTA

According to some embodiments, the preferred ratio of MTL to EDTA is about 1.00 MTL to about 0.15 EDTA. This preferred ratio balances MTL solubility and stability. According to the preferred embodiment, the ratio of MTL to EDTA is between 13:1 to 3:2 to maintain the Montelukast soluble within the film and prevent precipitation.

### MTL Solubility and EDTA: Application of Alkaline Buffering Agents

The final strategy used to incorporate more EDTA into the blend to improve stability while maintaining MTL solubility, is the incorporation of alkaline buffering components. An alkaline buffer will react with any free protons from EDTA that would normally acidify the blend, thereby allowing more EDTA to be added without a change in pH. In general these mixtures were prepared by first making an appropriate buffer. The buffer used in our experiments was selected for use in maintaining alkaline environments; CHES.

TABLE 17
Buffering Agent to Maintain MTL Solubility

| | MTL | Buffer | EDTA | | |
|---|---|---|---|---|---|
| Sample | (g) | System | (g) | pH | Solubility of MTL |
| Control A | 1.125 | none | 0.087 | 8 | Solubilize, clear yellow |
| Control B | 1.125 | none | 0.150 | 8-9 | Solubilize, clear yellow |
| Control C | 1.125 | none | 0.225 | 8-9 | Precipitates MTL |
| 1 | 1.125 | CHES | 0.225 | 9.3 | Precipitates MTL |
| 2 | 1.125 | CHES | 0.500 | 9.3 | Precipitates MTL |

Results indicate that when using the CHES buffer which maintains the pH at 9.3, MTL does not solubilize even after overnight mixing.

Illustrative, but non-limiting, examples of a formulation used to prepare a MTL oral films with EDTA are shown in Tables 18-24.

TABLE 18
MTL08

| | | | Composition | Composition |
|---|---|---|---|---|
| Item # | Description | Function | (% wet w/w) | % dry (w/w) |
| A | Methanol | Solvent (will | 0.28 | — |
| | | be removed | | |
| | | during manu- | | |
| | | facturing) | | |
| B | Purified | Solvent (will | 78.29 | — |
| | Water | be removed | | |
| | | during manu- | | |
| | | facturing) | | |
| 1 | Starch | Filler | 1.78 | 8.88 |
| 2 | HPC SL | Film former | 8.22 | 41.04 |
| | | polymer | | |
| 3 | Xanthan gum | Thickener | 0.86 | 4.32 |
| 4 | Sucralose | Sweetener | 0.43 | 2.16 |
| 5 | Glycerol | Plasticizer | 1.82 | 9.07 |
| 6 | Montelukast | Active | 3.24 | 16.18 |
| | Sodium | | | |
| 7 | Ascorbic acid | Stabilizer | 0.01 | 0.05 |
| 8 | Methylparaben | Anti-microbial | 0.11 | 0.54 |
| | | agent | | |
| 9 | Titanium | Opacifier | 0.27 | 1.32 |
| | Dioxide | | | |
| 10 | Yellow #10 | Color | 0.28 | 1.37 |
| 11 | HPC LF | Film former | 0.72 | 3.58 |
| | | polymer | | |
| 12 | Calcium | pH Modifier | 0.50 | 2.50 |
| | Carbonate | | | |
| 13 | Sodium | Permeation | 1.44 | 7.21 |
| | glycocholate | Enhancer | | |
| | Disodium | Stabilizer | 0.36 | 1.78 |
| | Edetate | | | |
| Total | | | 100 | 100.00 |

TABLE 19
MTL09

| | | | Composition | Composition |
|---|---|---|---|---|
| Item # | Description | Function | (% wet w/w) | % dry (w/w) |
| A | Methanol | Solvent (will | 0.2 | |
| | | be removed | | |
| | | during manu- | | |
| | | facturing) | | |
| B | Purified | Solvent (will | 79.68 | |
| | Water | be removed | | |
| | | during manu- | | |
| | | facturing) | | |
| 1 | Povidone | Film former | 10.89 | 54.12 |
| | | polymer | | |
| 2 | Locust Bean | Thickener | 0.86 | 4.30 |
| | Gum | | | |
| 3 | PEG 300 | Plasticizer | 0.15 | 0.73 |
| 4 | Labrafil | Permeation | 0.87 | 4.35 |
| | M1944CS | Enhancer | | |
| 5 | Sucralose | Sweetener | 0.43 | 2.15 |
| 6 | Citric Acid | pH Modifier | 0.60 | 2.98 |
| 7 | Montelukast | Active | 3.24 | 16.12 |
| | Sodium | | | |
| 8 | Disodium | Stabilizer | 0.36 | 1.78 |
| | Edetate | | | |
| 9 | Propylparaben | Anti-microbial | 0.10 | 0.49 |
| | | agent | | |
| 10 | Titanium | Opacifier | 0.27 | 1.32 |
| | Dioxide | | | |
| 11 | Yellow #10 | Color | 0.28 | 1.37 |
| 12 | HPC-GXF | Film former | 2.07 | 10.31 |
| | | polymer | | |
| Total | | | 100.00 | 100.00 |

TABLE 20
MTL10

| | | | Composition | Composition |
|---|---|---|---|---|
| Item # | Description | Function | (% wet (w/w) | % dry (w/w) |
| A | Methanol | Solvent (will | 0.29 | — |
| | | be removed | | |
| | | during manu- | | |
| | | facturing) | | |
| B | Purified | Solvent (will | 79.68 | — |
| | Water | be removed | | |
| | | during manu- | | |
| | | facturing) | | |
| 1 | Starch | Filler | 1.78 | 8.88 |
| 2 | Pullulan | Film former | 8.22 | 41.04 |
| | | polymer | | |
| 3 | Tara gum | Viscosity | 0.86 | 4.32 |
| | | Modifier | | |
| 4 | PEG 300 | Plasticizer | 0.15 | 0.74 |
| 5 | Sorbitol P60W | Plasticizer | 1.80 | 8.97 |
| 6 | Sucralose | Sweetener | 0.43 | 2.16 |
| 7 | Glycerol | Plasticizer | 1.82 | 9.07 |
| 8 | Montelukast | Active | 3.24 | 16.18 |
| | Sodium | | | |
| 9 | BHT | Stabilizer | 0.01 | 0.05 |
| 10 | Propylparaben | Anti-microbial | 0.11 | 0.54 |
| | | agent | | |
| 11 | Titanium | Opacifier | 0.27 | 1.32 |
| | Dioxide | | | |
| 12 | Yellow #10 | Color | 0.28 | 1.37 |
| 13 | HPC LF | Film former | 0.72 | 3.58 |
| | | polymer | | |
| 14 | Disodium | Stabilizer | 0.36 | 1.78 |
| | edetate | | | |
| Total | | | 100.00 | 100.00 |

TABLE 21
MTL11

| | | | Composition | Composition |
|---|---|---|---|---|
| Item # | Description | Function | (% wet (w/w) | % dry (w/w) |
| A | Methanol | Solvent (will | 0.29 | — |
| | | be removed | | |
| | | during manu- | | |
| | | facturing) | | |
| B | Purified | Solvent (will | 79.68 | — |
| | Water | be removed | | |
| | | during manu- | | |
| | | facturing) | | |
| 1 | Starch | Filler | 0.73 | 3.63 |
| 2 | PEO 200K | Film former | 8.22 | 41.04 |
| | | polymer | | |
| 3 | PEO 100K | Film former | 2.31 | 11.52 |
| | | polymer | | |
| 4 | Menthol | Flavor | 1.28 | 6.37 |
| 5 | Sorbitol P60W | Plasticizer | 1.65 | 8.24 |
| 6 | Sucralose | Sweetener | 0.43 | 2.16 |
| 7 | Citric Acid | pH Modifier | 0.44 | 2.21 |
| 8 | Montelukast | Active | 3.24 | 16.18 |
| | Sodium | | | |
| 9 | Sodium Sulfite | Stabilizer | 0.01 | 0.05 |
| 10 | Methylparaben | Anti-microbial | 0.11 | 0.54 |
| | | agent | | |
| 11 | Titanium | Opacifier | 0.27 | 1.32 |
| | Dioxide | | | |
| 12 | Yellow #10 | Color | 0.28 | 1.37 |
| 13 | HPC JF | Film former | 0.72 | 3.58 |
| | | polymer | | |
| 14 | Disodium | Stabilizer | 0.36 | 1.78 |
| | Edetate | | | |
| Total | | | 100.00 | 100.00 |

TABLE 22
MTL12

| | | | Composition | Composition |
|---|---|---|---|---|
| Item # | Description | Function | (% wet (w/w) | % dry (w/w) |
| A | Methanol | Solvent (will | 0.29 | — |
| | | be removed | | |
| | | during manu- | | |
| | | facturing) | | |
| B | Purified | Solvent (will | 78.66 | — |
| | Water | be removed | | |
| | | during manu- | | |
| | | facturing) | | |
| 1 | Sodium | pH Modifier | 0.64 | 3.03 |
| | Hydroxide | | | |
| 2 | HPMC E5 | Film former | 3.15 | 14.98 |
| | | polymer | | |
| 3 | HPC-L | Film former | 9.46 | 44.93 |
| | | polymer | | |
| 4 | PEG 300 | Plasticizer | 0.15 | 0.70 |
| 5 | Sorbitol P60W | Plasticizer | 1.80 | 8.54 |
| 6 | Sucralose | Sweetener | 0.43 | 2.05 |
| 7 | Sodium | Stabilizer | 0.58 | 2.75 |
| | Metabisulfite | | | |
| 8 | Montelukast | Active | 3.24 | 15.40 |
| | Sodium | | | |
| 9 | Disodium | Stabilizer | 0.38 | 1.83 |
| | Edetate | | | |
| 10 | Propylparaben | Anti-microbial | 0.11 | 0.51 |
| | | agent | | |
| 11 | Yellow #10 | Color | 0.28 | 1.31 |
| 12 | Oleic acid | Permeation | 0.83 | 3.97 |
| | | Enhancer | | |
| Total | | | 100.00 | 100.00 |

TABLE 23
MTL13

| | | | Composition | Composition |
|---|---|---|---|---|
| Item # | Description | Function | (% wet (w/w) | % dry (w/w) |
| A | Methanol | Solvent (will | 0.29 | — |
| | | be removed | | |
| | | during manu- | | |
| | | facturing) | | |
| B | Purified | Solvent (will | 79.68 | — |
| | Water | be removed | | |
| | | during manu- | | |
| | | facturing) | | |
| 1 | Sodium | pH Modifier | 0.83 | 4.12 |
| | Hydroxide | | | |
| 2 | Pullulan | Film former | 9.17 | 45.80 |
| | | polymer | | |
| 3 | Xanthan gum | Thickener | 1.85 | 9.22 |
| 4 | PEG 300 | Plasticizer | 0.15 | 0.74 |
| 5 | Sodium sulfite | Stabilizer | 0.64 | 3.19 |
| 6 | Sucralose | Sweetener | 0.43 | 2.16 |
| 7 | Glycerol | Plasticizer | 1.82 | 9.07 |
| 8 | Montelukast | Active | 3.24 | 16.18 |
| | Sodium | | | |
| 9 | Azone | Permeation | 0.90 | 4.51 |
| | | Enhancer | | |
| 10 | Propylparaben | Anti-microbial | 0.11 | 0.54 |
| | | agent | | |
| 11 | Titanium | Opacifier | 0.27 | 1.32 |
| | Dioxide | | | |
| 12 | Yellow #10 | Color | 0.28 | 1.37 |
| 13 | Disodium | Stabilizer | 0.36 | 1.78 |
| | Edetate | | | |
| Total | | | 100.00 | 100.00 |

TABLE 24
MTL14

| | | | Composition | Composition |
|---|---|---|---|---|
| Item # | Description | Function | % wet (w/w) | % dry (w/w) |
| A | Methanol | Solvent (will | 0.29 | — |
| | | be removed | | |
| | | during manu- | | |
| | | facturing) | | |
| B | Purified | Solvent (will | 79.68 | — |
| | Water | be removed | | |
| | | during manu- | | |
| | | facturing) | | |
| 1 | Ascorbic acid | Stabilizer | 0.95 | 4.76 |
| 2 | HPC-SL | Film former | 9.49 | 47.37 |
| | | polymer | | |
| 3 | Xanthan gum | Thickener | 1.40 | 7.01 |
| 4 | PEG 300 | Plasticizer | 0.15 | 0.74 |
| 5 | Sorbitol P60W | Plasticizer | 1.80 | 8.97 |
| 6 | Sucralose | Sweetener | 0.43 | 2.16 |
| 7 | Labrafil | Permeation | 1.00 | 5.00 |
| | M1944CS | Enhancer | | |
| 8 | Montelukast | Active | 3.24 | 16.18 |
| | Sodium | | | |
| 9 | Sodium | Stabilizer | 0.83 | 4.12 |
| | metabisulfite | | | |
| 10 | Propylparaben | Anti-microbial | 0.11 | 0.54 |
| | | agent | | |
| 11 | Yellow #10 | Color | 0.28 | 1.37 |
| 12 | Disodium | Stabilizer | 0.36 | 1.78 |
| | Edetate | | | |
| Total | | | 100.00 | 100.00 |

The surface pH of each formulation was measured (Table 25).

TABLE 25
Surface pH of MTL oral film

| Formulation | Montelukast state | Surface pH |
|---|---|---|
| MTL01 | Solubilized | 8.51 |
| MTL02 | Precipitate | 5.23 |
| MTL03 | Solubilized | 8.80 |
| MTL04 | Precipitate | 6.73 |
| MTL05 | Solubilized | 10.44 |
| MTL06 | Solubilized | 11.42 |
| MTL07 | Partially Solubilized | 7.38 |
| MTL08 | Solubilized | 8.25 |
| MTL09 | Precipitate | 4.98 |
| MTL10 | Solubilized | 8.52 |
| MTL11 | Precipitate | 5.81 |
| MTL12 | Solubilized | 10.26 |
| MTL13 | Solubilized | 11.26 |
| MTL14 | Partially Solubilized | 7.14 |

Formulations MTL01, MTL03 MTL05 MTL06 MTL07 MTL08 MTL10 MTL12 MTL13 and MTL14 are believed to be suitable for maintaining at least a portion of the MTL under a solubilized form within the film and improve the bioavailability of the Montelukast oral film when compared with Singulair® swallowable or chewable tablets. MTL02, MTL 03, MTL 09 and MTL11 provide a less desired dosage form in which the Montelukast precipitates and hence does not provide the desired improved bioavailability derived from alkaline surface pH.

The above description is considered that of the preferred embodiment(s) only. Modifications of these embodiments will occur to those skilled in the art and to those who make or use the illustrated embodiments. Therefore, it is understood that the embodiment(s) described above are merely exemplary and not intended to limit the scope of this disclosure, which is defined by the following claims as interpreted according to the principles of patent law, including the doctrine of equivalents.

1-31. (canceled)

32. An oral film dosage form, comprising:
a film layer having an alkaline surface pH from 7.5 to 9.5;
a safe and effective amount of Montelukast in a solubilized form incorporated into the film layer; and
EDTA, in a ratio of Montelukast to EDTA from 32:1 to 3:2, such that the Montelukast is maintained in the solubilized form;
wherein the film is buccally, orally or sublingually solubilized within 2-10 minutes.

33. The oral film dosage form of claim 32, wherein the film layer has a surface pH from 8 to 9.5.

34. The oral film dosage form of claim 32, wherein the film layer has a surface pH from 8.5 to 9.5.

35. The oral film dosage form of claim 32, wherein the film layer has a surface pH from 8 to 9.

36. The oral film dosage form of claim 32, wherein the film layer is unbuffered.

37. The oral film dosage form of claim 32, wherein the ratio of Montelukast to EDTA is about 1:0.15.

38. The oral film dosage form of claim 32, wherein the Montelukast is incorporated into the film layer in an amorphous form

39. The oral film dosage form of claim 32, wherein the Montelukast precipitates when the film layer dissolves and/or disintegrates in saliva.

40. The oral film dosage form of claim 32, wherein the film is buccally, orally or sublingually solubilized within 3-7 minutes and wherein the Montelukast precipitates in saliva.

41. The oral film dosage form of claim 32, wherein the Montelukast is present in an amount of about 0.5 mg to about 25 mg.

42. The oral film dosage form of claim 32, wherein the leukotriene receptor antagonist is Montelukast is present in an amount of about 5 mg to about 15 mg.

43. The oral film dosage form of claim 32, wherein the film layer is a bioadhesive film layer.

44. The oral film dosage form of claim 32, wherein the film is 80% solubilized within 1 minute when pre-dissolved in simulated saliva.

45. The oral film dosage form of claim 32, wherein the area under the curve (AUC) is between about 3120 and about 4700 ng*h/mL.

46. The oral film dosage form of claim 32, wherein the Cmax is between about 475 and about 720 ng/ml.

47. A multiple layer oral film dosage form, comprising:
a first film layer having an alkaline surface pH from 7.5 to 9.5 and containing a safe and effective amount of Montelukast in a solubilized form and EDTA in a ratio of Montelukast to EDTA from 32:1 to 3:2 such that the Montelukast is maintained in the solubilized form; and
at least a second film layer having a composition that is different from that of the first layer.

48. The multiple layer oral film dosage form of claim 47, wherein the second film layer is a non-adhesive barrier layer that prevents or reduces ingestion of the Montelukast.

49. The multiple layer oral film dosage form of claim 47, wherein the second film layer is formulated for enteral delivery of an active agent different from the Montelukast in the first film layer.

50. The multiple layer oral film dosage form of claim 47, wherein the second film layer comprises a taste-masking agent.

51. The multiple layer oral film dosage form of claim 47, wherein the second film layer comprises an active agent, the same or different from the Montelukast in the first film layer, wherein the second layer is formulated to provide a controlled release profile.

52. The oral film dosage form of claim 32, wherein the ratio of Montelukast to EDTA is about 16:1.

53. The oral film dosage form of claim 32, wherein the ratio of Montelukast to EDTA is about 13:1.

54. The oral film dosage form of claim 53, wherein the film is buccally, orally or sublingually solubilized within 2-7 minutes.

55. The multiple layer oral film dosage form of claim 47, wherein the ratio of Montelukast to EDTA is about 16:1.

56. The multiple layer oral film dosage form of claim 47, wherein the ratio of Montelukast to EDTA is about 13:1.
